(19) United States
(12) Patent Application Publication
(10) Pub. No.: US 2024/0277821 A1
(43) Pub. Date: Aug. 22, 2024

(54) TARGETING OF ANTIGEN-PRESENTING CELLS BY NANOPARTICLES CONTAINING POLYOXAZOLINE-LIPID CONJUGATES

(71) Applicant: Serina Therapeutics, Inc., Hunstville, AL (US)

(72) Inventors: Randall Moreadith, Huntsville, AL (US); J Milton Harris, Huntsville, AL (US); James Dahlman, Atlanta, GA (US); David Loughrey, Atlanta, GA (US); Kalina Paunovska, Atlanta, GA (US); Alejandro Da Silva Sanchez, Atlanta, GA (US)

(73) Assignees: Serina Therapeutics, Inc., Huntsville, AL (US); Georgia Tech Research Corporation, Altanta, GA (US)

(21) Appl. No.: 18/391,869

(22) Filed: Dec. 21, 2023

Related U.S. Application Data
(60) Provisional application No. 63/440,210, filed on Jan. 20, 2023.

(51) Int. Cl. A61K 39/00

(52) U.S. Cl. CPC A61K 39/00; A61K 2039/53; A61K 2039/545; A61K 2039/55555; A61K 2039/6018

(57) ABSTRACT

A method of targeting antigen-presenting cells and delivering an encapsulated payload with lipid nanoparticles including a POZ-lipid conjugate. The encapsulated payload may include, but is not limited to, a nucleic acid payload such as mRNA or modified mRNA. These LNPs are not subject to accelerated blood clearance and they have a low or reduced immunogenicity profile in vivo.

### FIELD OF THE INVENTION

The present disclosure relates to a method of targeting antigen-presenting cells with lipid nanoparticles (LNPs) including polyoxazoline-lipid conjugates (or pharmaceutical compositions including such LNPs). In addition, the LNPs of the present disclosure have a low (or reduced as compared to their PEG-LNP counterparts) immunogenicity profile in vivo.

### BACKGROUND OF THE INVENTION

Nucleic-acid (particularly mRNA)-based vaccines offer advantages over other vaccine technologies. Nucleic acid-based vaccines can be rapidly produced with reduced development time and costs by using a common manufacturing platform and purification methods regardless of the encoded antigen. In addition, mRNA-based vaccines only need to be delivered to the cytosol of the appropriate antigen-presenting cells to access the ribosomal translation machinery.

However, nucleic acid-based vaccines suffer from several shortcomings. For example, mRNA is rapidly degraded by nucleases in the body and is not readily taken up by most cell types. A key factor hampering both DNA and mRNA vaccine development is the lack of a potent, well-tolerated delivery system.

Efforts to address these shortcomings have resulted in encapsulation of mRNA payloads (as well as other oligonucleotide payloads) into lipid nanoparticles (LNPs), which protects mRNA from enzymatic degradation and enhances cell uptake and expression by up to 1000-fold compared to mRNA complexed to a polyamine. Such LNPs are typically composed of an ionizable lipid (which complexes with the oligonucleotide), cholesterol (to provide flexibility to the lipid bilayer), a lipid that includes a polyethylene glycol (PEG) moiety (to stabilize the lipid nanoparticles and prevent fusion with other nanoparticles), and a helper lipid (to provide structural integrity) such as distearoylphosphatidylcholine (DSPC). For example, U.S. Patent Publication No. 2020/0230058 discloses a liposome within which RNA encoding an immunogen of interest is encapsulated, where the liposome includes at least one PEG-lipid and where the PEG is present on the liposome's exterior and has an average molecular mass between 1 kDa and 3 kDa.

Early work with small interfering RNA (siRNA) identified the ionizable lipid as the primary driver of potency. Ionizable lipids are critically important for endosomal escape once the LNP is trafficking through the endosomal compartments in the cell.

LNPs are generally considered to be biocompatible nanocarriers with an excellent safety profile and capacity to carry both lipophilic and hydrophilic payloads. However, as briefly noted above, issues regarding immunogenicity of LNPs when administered to humans and animals exist. In fact, components of the LNP, such as PEG, may play a role in vaccine potency due to the impact of anti-PEG responses. It is increasingly recognized that treating patients with PEGylated components, including lipids, can lead to the formation of antibodies that specifically recognize and bind to PEG (i.e., anti-PEG antibodies). Anti-PEG antibodies are also found in patients who have never been treated with PEGylated drugs but have been exposed to products containing PEG.

Consequently, treating patients who have acquired anti-PEG antibodies with LNPs containing PEGylated lipids may result in accelerated blood clearance of LNPs, low drug efficacy, hypersensitivity, and, in some cases, life-threatening side effects, including allergic reactions known as anaphylaxis. This immunogenicity of PEG may cause a particular problem when the subject receives repeated vaccinations over time with LNPs containing PEGylated lipids or if the subject has been previously exposed to products containing PEG.

Accordingly, there is a need in the art for solutions to address the shortcomings of current LNP technology. Indeed, the art is particularly in need of LNP formulations with reduced or absent immunogenicity after initial administration of the LNP and after subsequent administration of the LNP. In addition, it would be advantageous to provide a LNP with an encapsulated payload that allows preferential targeting of the antigen-presenting cells, particularly as it may relate to vaccines for infectious diseases and cancer immunotherapy. The present disclosure provides such a solution.

### SUMMARY OF THE INVENTION

The utility of lipid nanoparticles (LNP) for delivering mRNA to cells has recently been demonstrated in vaccines for Covid-19 (or SARS-COV-2). Given the millions of deaths resulting from this viral disease and given the apparent wide utility of cellular delivery of mRNA for a range of diseases from cancer to influenza, there has been a substantial amount of work on understanding and improving LNP delivery of mRNA. As part of this research, the inventors have been examining polyoxazoline (POZ) conjugates with lipids (POZ-lipids) as a replacement for polyethylene glycol (PEG) conjugates of lipids (PEG-lipids) that are currently used in the commercially available mRNA vaccines. PEG-lipids have a number of disadvantages including immunogenicity and accompanying adverse immune reactions in patients, and storage conditions requiring extremely low-temperature conditions to maintain stability.

The present disclosure relates to nanoparticles including polyoxazoline (POZ)-lipids used to target antigen-presenting cells. In particular, the inventors have now made the surprising observation that LNPs made with POZ-lipids (as compared with PEG-lipids) provide preferential uptake by the antigen-presenting cells of the spleen critical to developing an immune response. These antigen-presenting cells are the macrophage and dendritic cells that ultimately lead to antibody production and generation of cytotoxic T cells that are vital to vaccine function. This preferential uptake or targeting by the LNPs of the present disclosure may provide an enhanced vaccine response. As such, a lower dose of the vaccine may be used to produce an optimal protective antibody response. This lower dose, in turn, can be expected to provide a reduction in side effects from vaccine injection.

Without being bound by any particular theory, this uptake by antigen-presenting cells is independent of formulation. More specifically, the inventors have discovered uptake for certain LNP compositions including a POZ-lipid LNP modeled after the commercially available Moderna® Spikevax COVID-19 vaccine (currently delivered with a PEG-lipid LNP). The inventors have discovered similarly effective preferential uptake in a POZ-lipid LNP modeled after the commercially available Alnylam® neuropathy drug marketed as Onpattro® (currently delivered with a PEG-lipid LNP). It is contemplated that the targeted delivery of POZ-lipid-containing LNPs will provide highly efficacious methods to improve upon a range of oligonucleotide therapeutics including vaccines, cancer immunotherapy, and gene therapy.

The present disclosure relates to a method for preferentially delivering mRNA to antigen-presenting cells including providing a lipid nanoparticle including a POZ-lipid of Formula I:

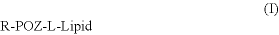

wherein R includes an initiating group, POZ comprises poly(oxazoline), L includes a physiologically degradable linking group, and Lipid includes a non-charged lipid including at least one hydrophobic moiety. In some embodiments, POZ is poly(ethyloxazoline).

In some embodiments, the antigen-presenting cells include macrophage cells, dendritic cells, or combinations thereof. In other embodiments, POZ includes [N(COR₂)CH₂CH₂]ₙ, where R₂ is ethyl. In still other embodiments, R includes a hydrogen, a substituted or unsubstituted alkyl, an alkyne-substituted alkyl, a triazole with attached carboxylic acid, or a substituted or unsubstituted aralkyl group. In yet other embodiments, L includes ethers, esters, carboxylate esters, carbonate esters, carbamates, amines, amides, urethanes, disulfides, and combinations thereof. In still other embodiments, Lipid includes two hydrophobic moieties. In other embodiments, Lipid includes phospholipid, a glycerolipid, a di-alkyl acetamide, or a combination thereof. In yet other embodiments, Lipid includes 1,2-dimyristoyl-sn-glycerol, 1,2-dilauroyl-sn-glycerol, or a combination thereof.

The present disclosure also relates to a method for preferentially delivering mRNA to antigen-presenting cells including providing a lipid nanoparticle including a POZ-lipid of Formula II:

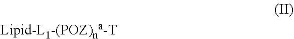

wherein Lipid includes a non-charged lipid including at least one hydrophobic moiety, L₁ includes a physiologically degradable linking group, POZ includes a polyoxazoline polymer of the structure [N(COR₂)CH₂CH₂], wherein R₂ is ethyl, n ranges from 1 to 1,000, a is ran, which indicates a random copolymer, or block, which indicates a block copolymer, and T includes a terminating group.

In some embodiments, L₁ includes ethers, esters, carboxylate esters, carbonate esters, carbamates, amines, amides, urethanes, disulfides, and combinations thereof. In other embodiments, L₁ includes a triazole.

In yet other embodiments, T includes Z-B-Q, and wherein Z includes S, O, or N, B is an optional linking group, and Q is a terminating nucleophile or portion thereof. In still other embodiments, Lipid includes two hydrophobic moieties. In other embodiments, Lipid includes a phospholipid, a glycerolipid, a di-alkyl acetamide, or a combination thereof. In still other embodiments, Lipid includes 1,2-dimyristoyl-sn-glycerol, 1,2-dilauroyl-sn-glycerol, or a combination thereof.

The present disclosure relates to a method for preferentially delivering mRNA to antigen-presenting cells including providing a lipid nanoparticle including a POZ-lipid of Formula III:

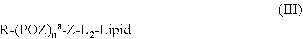

wherein R includes an initiating group; POZ comprises a polyoxazoline polymer of the structure [N(COR₂)CH₂CH₂], wherein R₂ is ethyl; n ranges from 1 to 1,000; a is ran, which indicates a random copolymer, or block, which indicates a block copolymer; Z comprises S, O, or N; L₂ includes a physiologically degradable linking group, and Lipid includes a non-charged lipid comprising at least one hydrophobic group.

In some embodiments, L₂ includes ethers, esters, carboxylate esters, carbonate esters, carbamates, amines, amides, urethanes, disulfides, and combinations thereof. In other embodiments, Lipid includes two hydrophobic moieties. In still other embodiments, Lipid includes a phospholipid, a glycerolipid, a di-alkyl acetamide, or a combination thereof. In yet other embodiments, Lipid includes 1,2-dimyristoyl-sn-glycerol, 1,2-dilauroyl-sn-glycerol, or a combination thereof. In still other embodiments, R includes a hydrogen, or a substituted or unsubstituted alkyl, and n ranges from 15 to 35.

The present disclosure relates to a method for preferentially delivering mRNA to antigen-presenting cells including providing a lipid nanoparticle including a POZ-lipid of Formula IV:

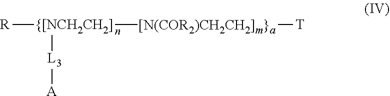

wherein R includes an initiating group, L₃ includes a physiologically degradable linking group, Lipid includes a non-charged lipid comprising at least one hydrophobic moiety, n ranges from 1 to 5, R₂ is independently selected for each repeating unit from an unsubstituted or substituted alkyl, alkenyl, aralkyl, heterocyclylalkyl, or active functional group, m ranges from 1 to 100, a is ran, which indicates a random copolymer, or block, which indicates a block copolymer, and T includes a terminating group.

In some embodiments, L₃ includes esters, carboxylate esters, carbonate esters, carbamates, amides, and combinations thereof. In other embodiments, L₃ includes a triazole. In still other embodiments, T includes Z-B-Q, and wherein Z includes S, O, or N, B is an optional linking group, and Q is a terminating nucleophile or portion thereof. In yet other embodiments, Lipid includes two hydrophobic moieties. In still other embodiments, Lipid includes a phospholipid, a glycerolipid, a di-alkyl acetamide, or a combination thereof. In yet other embodiments, Lipid includes 1,2-dimyristoyl-sn-glycerol, 1,2-dilauroyl-sn-glycerol, or a combination thereof.

In some embodiments, the LNPs of the present disclosure encapsulate a payload and wherein the subject receives an effective amount of the payload for treating a condition. In this aspect, the LNPs of the present disclosure allow for delivery of an encapsulated payload to target tissues of a subject while producing a reduced immune response that allows repeat doses of the LNP. The method involves administering a first dose of LNPs to the subject, wherein the first dose of LNPs produces an attenuated immune response of a subsequent dose of LNP, and administering a subsequent dose of LNPs to the subject, wherein the subject has an attenuated ABC response to the subsequent dose of LNPs.

In other embodiments, the method of the present disclosure involves administering to a subject a first dose of LNPs of the present disclosure, which encapsulate an mRNA coding for an antigen or protein of interest, wherein the first dose of LNPs induces an attenuated immune response that lessens the accelerated blood clearance (ABC) upon administration of a subsequent dose of LNP. In some aspects, the LNP of the present disclosure becomes insensitive to accelerated blood clearance upon repeated administration in vivo.

### BRIEF DESCRIPTION OF THE DRAWINGS

Further features and advantages can be ascertained from the following detailed description that is provided in connection with the drawings described below:

FIG. 1A is a graphical representation of the hydrodynamic diameters of SM-102-containing LNPs formulated with PEG 2000 and DMG-PEOZ 2000;

FIG. 1B is a graphical representation of the hydrodynamic diameters of MC3-containing LNPs formulated with PEG 2000 and PEOZ 2000;

FIG. 2A is a graphical representation of the polydispersity indices of SM-102-containing LNPs formulated with PEG 2000 and DMG-PEOZ 2000;

FIG. 2B is a graphical representation of the polydispersity indices of MC3-containing LNPs formulated with PEG 2000 and PEOZ 2000;

FIG. 3A is a graphical representation of the polydispersity indices of SM-102-containing LNPs formulated with PEG 2000 and DMG-PEOZ 2000;

FIG. 3B is a graphical representation of the encapsulation efficiencies of MC3-containing LNPs formulated with PEG 2000 and PEOZ 2000;

FIG. 4 is a graphical representation of the uptake and expression of aVHH+ by cells in the liver and spleen after intravenous administration of SM-102-containing LNPs formulated with PEG 2000 and PEOZ 2000;

FIG. 5 is a graphical representation of the uptake and expression of aVHH+ by cells in the liver and spleen after intravenous administration of MC3-containing LNPs formulated with PEG 2000 and PEOZ 2000;

FIG. 6 is a graphical representation of the uptake and expression of aVHH+ by cells in muscle and lumbar aortic lymph nodes after intramuscular administration of SM-102-containing LNPs formulated with PEG 2000 and PEOZ 2000;

FIG. 7 is a graphical representation of the uptake and expression of aVHH+ by cells in the liver and spleen after intramuscular administration of DMG-containing LNPs formulated with PEG 2000 and PEOZ 2000;

FIG. 8A is a graphical representation of the bioluminescence in the liver after repeat intramuscular administration of SM-102-containing LNPs formulated with PEG and POZ lipid conjugates;

FIG. 8B is a graphical representation of the bioluminescence in the spleen after repeat intramuscular administration of SM-102-containing LNPs formulated with PEG and POZ lipid conjugates; and

FIG. 9 is a graphical representation of the anti-PEG and anti-PEOZ IgM after intramuscular administration of SM-102-containing LNPs formulated with PEG and POZ lipid conjugates.

### DETAILED DESCRIPTION

The present disclosure provides a method of targeting antigen-presenting cells with lipid nanoparticles (LNPs) including a POZ-lipid conjugate. The LNPs may be used to preferentially deliver an encapsulated payload, e.g., a nucleic acid payload including, but not limited to, mRNA or modified mRNA, to macrophage and dendritic cells. Because LNPs including a POZ-lipid conjugate of the present disclosure have no immunogenicity, or reduced immunogenicity, as compared to a corresponding LNP containing a PEG-lipid, such LNPs are believed to not only provide higher efficacy, but also provide a safer method of delivering nucleic acid vaccines.

LNPs of the prior art generally include a cationic or ionizable lipid combined with: (i) a helper lipid that supports the bilayer structure and facilitates the endocytosis; (ii) a sterol lipid (i.e., cholesterol) to stabilize the lipid bilayer of the LNP; and (iii) a PEG-lipid to provide the LNP with a hydrating layer to improve colloidal stability, prevent fusion of nascent particles, reduce protein adsorption and non-specific uptake, and prevent reticuloendothelial clearance. However, as mentioned above, the PEG-lipid in these LNPs may compromise patient safety due to the potential of anti-PEG immune responses. Using a LNP including a POZ-lipid in accordance with the present disclosure provides a highly efficacious method to deliver nucleic acids and avoid the immunogenicity issues typically associated with PEGylated LNPs.

### Definitions

All patent applications, patents, and printed publications cited herein are incorporated herein by reference in the entireties, except for any definitions, subject matter disclaimers or disavowals, and except to the extent that the incorporated material is inconsistent with the express disclosure herein, in which case the language in this disclosure controls.

The articles “a” and “an” are used herein to refer to one or to more than one (i.e., to at least one) of the grammatical object of the article. By way of example, “an element” means one element or more than one element.

The terms “about” and “approximately” shall generally mean an acceptable degree of error or variation for the quantity measured given the nature or precision of the measurements. Numerical quantities given in this description are approximate unless stated otherwise, meaning that the term “about” or “approximately” can be inferred when not expressly stated.

As used herein, the term “active” or “activated” when used in conjunction with a particular functional group refers to a functional group that reacts readily with an electrophile or a nucleophile on another molecule. This is in contrast to those groups that require catalysts or impractical reaction conditions in order to react (i.e., a “non-reactive” or “inert” group).

As used herein, the term “attenuated” when used in conjunction with a drug or immune response, including, but not limited to, induction of IgM, induction of IgG, accelerated blood clearance, or combination thereof, refers to a lesser or weakened response, presence, or activity as compared to the response, presence, or activity prior to administration of the compound/composition.

As used herein, the term “physiologically degradable” or “physiologically releasable” refers to a linkage containing a cleavable moiety. The terms degradable and releasable do not imply any particular mechanism by which the linker is cleaved.

As used herein, the term “link”, “linked” “linkage” or “linker” when used with respect to a POZ polymer, POZ conjugate, an agent, or compound described herein, or components thereof, refers to bonds that normally are formed as the result of a chemical reaction and typically are covalent linkages.

As used herein, the term “lipid nanoparticle” or “LNP” is used to encompass any of the many types of nanoparticles, including liposomes, that are formed by a lipid layer or layers surrounding a core containing a molecule to be released into the body. Liposomes generally have one or more contiguous lipid bilayers encapsulating an aqueous core. Other forms of liposome-like nanocarriers may have a lipid monolayer, or a non-contiguous bilayer, and may or may not have an aqueous core.

As used herein, the term “hydrophilic,” for example with reference to a hydrophilic group, refers to a compound or molecule, or a portion thereof, where the interaction with water is thermodynamically more favorable than interaction with oil or other hydrophobic solvents. A hydrophilic compound is able to dissolve in, or be dispersed in, water.

As used herein, the term “hydrophobic”, for example with reference to a hydrophobic portion, refers to a compound or molecule, or a portion thereof, where the interaction with water is thermodynamically less favorable than interaction with oil or other hydrophobic solvents. A hydrophobic compound is able to dissolve in, or be dispersed in, oil or other hydrophobic solvents.

As used herein, the term “inert” or “non-reactive” when used in conjunction with a particular functional group refers to a functional group that does not react readily with an electrophile or a nucleophile on another molecule and require catalysts or impractical reaction conditions in order to react.

As used herein, the term “pendent group” refers to a part of the POZ polymer that is attached to the POZ polymer.

As used herein, the term “pendent moiety” refers to a substituent that is linked to the POZ polymer portion via a linking group; a pendent moiety is exemplified by R₂ of formula IV as described herein.

As used herein, the term “pharmaceutically acceptable” refers to a compound that is compatible with the other ingredients of a composition and not deleterious to the subject receiving the compound or composition. In some embodiments, the term “pharmaceutically acceptable” means approved by a regulatory agency of the Federal or a state government or listed in the U.S. Pharmacopeia or other generally recognized pharmacopeia for use in animals, and more particularly in humans.

As used herein, the term “pharmaceutically acceptable form” is meant to include known forms of a compound or POZ conjugate that may be administered to a subject, including, but not limited to, solvates, hydrates, prodrugs, isomorphs, polymorphs, pseudomorphs, neutral forms and salt forms of a compound. In certain embodiments, the pharmaceutically acceptable form excludes prodrugs, isomorphs and/or pseudomorphs. In certain embodiments, the pharmaceutically acceptable form is limited to pharmaceutically acceptable salts, neutral forms, solvates and hydrates. In certain embodiments, the pharmaceutically acceptable form is limited to pharmaceutically acceptable salts and neutral forms. In certain embodiments, the pharmaceutically acceptable form is limited to pharmaceutically acceptable salts.

As used herein, the term “alkyl”, whether used alone or as part of a substituent group, is a term of art and refers to saturated aliphatic groups that optionally contain one or more heteroatoms (such as O, S or N) which may be optionally substituted, including straight-chain alkyl groups, branched-chain alkyl groups, cycloalkyl groups, alkyl substituted cycloalkyl groups, and cycloalkyl substituted alkyl groups. In certain embodiments, a straight-chain or branched-chain alkyl has about 30 or fewer carbon atoms in its backbone (e.g., C₁-C₃₀ for straight chain, C₃-C₃₀ for branched chain), and alternatively, about 20 or fewer, or 10 or fewer. In certain embodiments, the term “alkyl” refers to a C₁-C₁₀ straight-chain alkyl group or a C₁-C₃ straight-chain alkyl group. In certain embodiments, the term “alkyl” refers to a C₃-C₁₂ branched-chain alkyl group. In certain embodiments, the term “alkyl” refers to a C₃-C₈ branched-chain alkyl group. Representative examples of alkyl include, but are not limited to, methyl, ethyl, n-propyl, iso-propyl, n-butyl, sec-butyl, iso-butyl, tert-butyl, n-pentyl, isopentyl, neopentyl, and n-hexyl. In certain embodiments, the term “alkyl” refers to a C₁-C₁₀ straight-chain alkyl group that contains one or more heteroatoms in place of a carbon atom (such as O, S or N), wherein the heteroatom may be optionally substituted. In certain embodiments, the term “alkyl” refers to a C₁-C₁₀ straight-chain alkyl group that is substituted with up to 5 groups selected from the group consisting of OH, NH₂ and ═O.

As used herein, the term “alkenyl”, whether used alone or as part of a substituent group, is a term of art and refers to unsaturated aliphatic groups that optionally contain one or more heteroatoms (such as O, S or N) which may be optionally substituted, including, a straight or branched chain hydrocarbon radical containing from 2 to 30 carbons and containing at least one carbon-carbon double bond formed by the removal of two hydrogens. Representative examples of alkenyl include, but are not limited to, ethenyl, 2-propenyl, 2-methyl-2-propenyl, 3-butenyl, 4-pentenyl, 5-hexenyl, 2-heptenyl, 2-methyl-1-heptenyl, and 3-decenyl. The unsaturated bond(s) of the alkenyl group can be located anywhere in the moiety and can have either the (Z) or the (E) configuration about the double bond(s).

As used herein, the term “alkynyl”, whether used alone or as part of a substituent group, is a term of art and refers to unsaturated aliphatic groups that optionally contain one or more heteroatoms (such as O, S or N) which may be optionally substituted, including, straight or branched chain hydrocarbon radical containing from 2 to 30 carbon atoms and containing at least one carbon-carbon triple bond. Representative examples of alkynyl include, but are not limited, to acetylenyl, 1-propynyl, 2-propynyl, 3-butynyl, 2-pentynyl, 4-pentynyl, and 1-butynyl.

As used herein, the term “substituted alkyl”, “substituted alkenyl”, and “substituted alkynyl” refers to alkyl, alkenyl and alkynyl groups as defined above in which one or more bonds to a carbon(s) or hydrogen(s) are replaced by a bond to non-hydrogen or non-carbon atoms such as, but not limited to, a halogen atom in halides such as F, Cl, Br, and I; and oxygen atom in groups such as carbonyl, carboxyl, hydroxyl groups, alkoxy groups, aryloxy groups, heterocyclyloxy groups, and ester groups; a sulfur atom in groups such as thiol groups, alkyl and aryl sulfide groups, sulfone groups, sulfonyl groups, and sulfoxide groups; a nitrogen atom in groups such as amines, amides, alkylamines, dialkylamines, arylamines, alkylarylamines, diarylamines, N-oxides, imides, enamines imines, oximes, hydrazones, heterocyclylamine, (alkyl)(heterocyclyl)-amine, (aryl)(heterocyclyl)amine, diheterocyclylamine, triazoles, and nitriles; a silicon atom in groups such as in trialkylsilyl groups, dialkylarylsilyl groups, alkyldiarylsilyl groups, and triarylsilyl groups; and other heteroatoms in various other groups. In a specific embodiment, a “polar alkyl”, “polar alkenyl”, and “polar alkynyl”, refers to alkyl, alkenyl, and alkynyl groups substituted with an atom that results in a polar covalent bond. In another specific embodiment, a “polar alkyl”, “polar alkenyl”, and “polar alkynyl” refers to C1 to C5 alkyl, alkenyl, and alkynyl, groups substituted with an atom that results in a polar covalent bond. In a specific embodiment, a “polar alkyl”, “polar alkenyl”, and “polar alkynyl”, refers to alkyl, alkenyl, alkynyl groups, such as C1 to C5 alkyl, alkenyl, and alkynyl groups, substituted with an —OH group and/or a —C(O)—OH group. As used herein, the term “halo” or “halogen” whether used alone or as part of a substituent group, is a term of art and refers to —F, —Cl, —Br, or —I.

As used herein, the term “alkoxy”, whether used alone or as part of a substituent group, is a term of art and refers to an alkyl group, as defined herein, appended to the parent molecular moiety through an oxygen atom. Representative examples of alkoxy include, but are not limited to, methoxy, ethoxy, propoxy, 2-propoxy, butoxy, tert-butoxy, pentyloxy, and hexyloxy.

As used herein, the term “aralkyl” or “arylalkyl”, whether used alone or as part of a substituent group, is a term of art and refers to an alkyl group substituted with an aryl group, wherein the moiety is appended to the parent molecule through the alkyl group. An arylalkyl group may be optionally substituted. A “substituted aralkyl” has the same meaning with respect to unsubstituted aralkyl groups that substituted aryl groups had with respect to unsubstituted aryl groups. However, a substituted aralkyl group also includes groups in which a carbon or hydrogen bond of the alkyl part of the group is replaced by a bond to a non-carbon or a non-hydrogen atom.

As used herein, the term “heteroaralkyl” or “heteroarylalkyl”, whether used alone or as part of a substituent group, is a term of art and refers to an alkyl group substituted with a heteroaryl group, wherein the moiety is appended to the parent molecular moiety through the alkyl group. A heteroarylalkyl may be optionally substituted. The term “substituted heteroarylalkyl” has the same meaning with respect to unsubstituted heteroarylalkyl groups that substituted aryl groups had with respect to unsubstituted aryl groups.

As used herein, the term “heterocyclylalkyl”, whether used alone or as part of a substituent group, is a term of art and refers to unsubstituted or substituted alkyl, alkenyl or alkynyl groups in which a hydrogen or carbon bond of the unsubstituted or substituted alkyl, alkenyl or alkynyl group is replaced with a bond to a heterocyclyl group. A heterocyclylalkyl may be optionally substituted. The term “substituted heterocyclylalkyl” has the same meaning with respect to unsubstituted heterocyclylalkyl groups that substituted aryl groups had with respect to unsubstituted aryl groups. However, a substituted heterocyclylalkyl group also includes groups in which a non-hydrogen atom is bonded to a heteroatom in the heterocyclyl group of the heterocyclylalkyl group such as, but not limited to, a nitrogen atom in the piperidine ring of a piperidinylalkyl group.

As used herein, the term “aryl”, whether used alone or as part of a substituent group, is a term of art and refers to includes monocyclic, bicyclic and polycyclic aromatic hydrocarbon groups, for example, benzene, naphthalene, anthracene, and pyrene. The aromatic ring may be substituted at one or more ring positions with one or more substituents, such as halogen, azide, alkyl, aralkyl, alkenyl, alkynyl, cycloalkyl, hydroxyl, alkoxyl, amino, nitro, sulfhydryl, imino, amido, phosphonate, phosphinate, carbonyl, carboxyl, silyl, ether, alkylthio, sulfonyl, sulfonamido, ketone, aldehyde, ester, heterocyclyl, aromatic or heteroaromatic moieties, fluoroalkyl (such as trifluromethyl), cyano, or the like. The term “aryl” also includes polycyclic ring systems having two or more cyclic rings in which two or more carbons are common to two adjoining rings (the rings are “fused rings”) wherein at least one of the rings is an aromatic hydrocarbon, e.g., the other cyclic rings may be cycloalkyls, cycloalkenyls, cycloalkynyls, aryls, heteroaryls, and/or heterocyclyls. In certain embodiments, the term “aryl” refers to a phenyl group. The aryl group may be optionally substituted.

As used herein, the term “cycloalkyl”, whether used alone or as part of a substituent group, is a term of art and refers to a saturated carbocyclic group containing from three to six ring carbon atoms, wherein such ring may optionally be substituted with a substituted or unsubstituted alkyl group or a substituent as described for a substituted alkyl group. Exemplary cycloalkyl groups include, but are not limited to, cyclopropyl, cyclobutyl, cyclopentyl, cyclohexyl, 2-methylcyclobutyl and 4-ethylcyclohexyl.

As used herein, the term “heteroaryl”, whether used alone or as part of a substituent group, is a term of art and refers to a monocyclic, bicyclic, and polycyclic aromatic group having 3 to 30 total atoms including one or more heteroatoms such as nitrogen, oxygen, or sulfur in the ring structure. Exemplary heteroaryl groups include azaindolyl, benzo(b)thienyl, benzimidazolyl, benzofuranyl, benzoxazolyl, benzothiazolyl, benzothiadiazolyl, benzotriazolyl, benzoxadiazolyl, furanyl, imidazolyl, imidazopyridinyl, indolyl, indolinyl, indazolyl, isoindolinyl, isoxazolyl, isothiazolyl, isoquinolinyl, oxadiazolyl, oxazolyl, purinyl, pyranyl, pyrazinyl, pyrazolyl, pyridinyl, pyrimidinyl, pyrrolyl, pyrrolo[2,3-d]pyrimidinyl, pyrazolo[3,4-d]pyrimidinyl, quinolinyl, quinazolinyl, triazolyl, thiazolyl, thiophenyl, tetrahydroindolyl, tetrazolyl, thiadiazolyl, thienyl, thiomorpholinyl, triazolyl or tropanyl, and the like. The “heteroaryl” may be substituted at one or more ring positions with one or more substituents such as halogen, azide, alkyl, aralkyl, alkenyl, alkynyl, cycloalkyl, hydroxyl, alkoxyl, amino, nitro, sulfhydryl, imino, amido, phosphonate, phosphinate, carbonyl, carboxyl, silyl, ether, alkylthio, sulfonyl, sulfonamido, ketone, aldehyde, ester, heterocyclyl, aromatic or heteroaromatic moieties, fluoroalkyl (such as trifluromethyl), cyano, or the like. The term “heteroaryl” also includes polycyclic ring systems having two or more cyclic rings in which two or more carbons are common to two adjoining rings (the rings are “fused rings”) wherein at least one of the rings is an aromatic group having one or more heteroatoms in the ring structure, e.g., the other cyclic rings may be cycloalkyls, cycloalkenyls, cycloalkynyls, aryls, heteroaryls, and/or heterocyclyls.

As used herein, the term “heterocyclyl”, whether used alone or as part of a substituent group, is a term of art and refers to a radical of a non-aromatic ring system, including, but not limited to, monocyclic, bicyclic, and tricyclic rings, which can be completely saturated or which can contain one or more units of unsaturation, for the avoidance of doubt, the degree of unsaturation does not result in an aromatic ring system, and having 3 to 15 atoms including at least one heteroatom, such as nitrogen, oxygen, or sulfur. For purposes of exemplification, which should not be construed as limiting the scope of this invention, the following are examples of heterocyclic rings: aziridinyl, azirinyl, oxiranyl, thiiranyl, thiirenyl, dioxiranyl, diazirinyl, diazepanyl, 1,3-dioxanyl, 1,3-dioxolanyl, 1,3-dithiolanyl, 1,3-dithianyl, imidazolidinyl, isothiazolinyl, isothiazolidinyl, isoxazolinyl, isoxazolidinyl, azetyl, oxetanyl, oxetyl, thietanyl, thietyl, diazetidinyl, dioxetanyl, dioxetenyl, dithietanyl, dithietyl, dioxalanyl, oxazolyl, thiazolyl, triazinyl, isothiazolyl, isoxazolyl, azepines, azetidinyl, morpholinyl, oxadiazolinyl, oxadiazolidinyl, oxazolinyl, oxazolidinyl, oxopiperidinyl, oxopyrrolidinyl, piperazinyl, piperidinyl, pyranyl, pyrazolinyl, pyrazolidinyl, pyrrolinyl, pyrrolidinyl, quinuclidinyl, thiomorpholinyl, tetrahydropyranyl, tetrahydrofuranyl, tetrahydrothienyl, thiadiazolinyl, thiadiazolidinyl, thiazolinyl, thiazolidinyl, thiomorpholinyl, 1,1-dioxidothiomorpholinyl (thiomorpholine sulfone), thiopyranyl, and trithianyl. A heterocyclyl group may be substituted at one or more ring positions with one or more substituents such as halogen, azide, alkyl, aralkyl, alkenyl, alkynyl, cycloalkyl, hydroxyl, alkoxyl, amino, nitro, sulfhydryl, imino, amido, phosphonate, phosphinate, carbonyl, carboxyl, silyl, ether, alkylthio, sulfonyl, sulfonamido, ketone, aldehyde, ester, heterocyclyl, aromatic or heteroaromatic moieties, fluoroalkyl (such as trifluromethyl), cyano, or the like.

As used herein, the terms “treatment”, “treat”, and “treating” refers a course of action (such as administering a conjugate as described herein or pharmaceutical composition comprising a conjugate as described herein) so as to prevent, eliminate, or reduce a symptom, aspect, or characteristics of a disease or condition. Such treating need not be absolute to be useful. In one embodiment, treatment includes a course of action that is initiated concurrently with or after the onset of a symptom, aspect, or characteristics of a disease or condition. In another embodiment, treatment includes a course of action that is initiated before the onset of a symptom, aspect, or characteristics of a disease or condition.

As used herein, the term “in need of treatment” refers to a judgment made by a caregiver that a patient requires or will benefit from treatment. This judgment is made based on a variety of factors that are in the realm of a caregiver's expertise, but that includes the knowledge that the patient is ill, or will be ill, as the result of a disease or condition that is treatable by a method or compound of the disclosure.

As used herein, the terms “individual”, “subject”, or “patient” refers to any animal, including mammals, such as mice, rats, other rodents, rabbits, dogs, cats, swine, cattle, sheep, horses, or primates, and humans. The terms may specify male or female or both, or exclude male or female. In a preferred embodiment, the terms “individual”, “subject”, or “patient” refers to a human.

As used herein, the term “therapeutically effective amount” refers to an amount of a conjugate, either alone or as a part of a pharmaceutical composition, that is capable of having any detectable, positive effect on any symptom, aspect, or characteristics of a disease or condition. Such effect need not be absolute to be beneficial.

It will be understood that “substitution” or “substituted with” includes the implicit proviso that such substitution is in accordance with permitted valence of the substituted atom and the substituent, and that the substitution results in a stable compound, e.g., which does not spontaneously undergo transformation such as by rearrangement, fragmentation, decomposition, cyclization, elimination, or other reaction.

It will be understood that when a group is specified as a part of a compound, the substitution of the group may be adjusted to accommodate the particular bonds. For example, when an alkyl group is joined to two other groups, the alkyl group is considered an alkylene group.

The term “substituted” is also contemplated to include all permissible substituents of organic compounds. In a broad aspect, the permissible substituents include acyclic and cyclic, branched and unbranched substituents, carbocyclic and heterocyclyl, aromatic and nonaromatic substituents of organic compounds. Illustrative substituents include, for example, those described herein above. For purposes of this disclosure, the heteroatoms, such as oxygen or nitrogen, may have hydrogen substituents and/or any permissible substituents of organic compounds described herein which satisfy the valences of the heteroatoms. Exemplary substitutions include, but are not limited to, hydroxy, halogen, azide, alkyl, aralkyl, alkenyl, alkynyl, cycloalkyl, hydroxyl, alkoxyl, amino, nitro, sulfhydryl, imino, amido, phosphonate, phosphinate, carbonyl, carboxyl, silyl, ether, alkylthio, sulfonyl, sulfonamido, ketone, aldehyde, ester, heterocyclyl, aromatic or heteroaromatic moieties, fluoroalkyl (such as trifluromethyl), cyano, or the like. This invention is not intended to be limited in any manner by the permissible substituents of organic compounds.

Other chemistry terms herein are used according to conventional usage in the art, as exemplified by The McGraw-Hill Dictionary of Chemical Terms (ed. Parker, S., 1985), McGraw-Hill, San Francisco, incorporated herein by reference). Unless otherwise defined, all technical and scientific terms used herein have the same meaning as commonly understood by one of ordinary skill in the art to which this invention pertains.

The term “pharmaceutically acceptable salt” as used herein includes salts derived from inorganic or organic acids including, for example, hydrochloric, hydrobromic, sulfuric, nitric, perchloric, phosphoric, formic, acetic, lactic, maleic, fumaric, succinic, tartaric, glycolic, salicylic, citric, methanesulfonic, benzenesulfonic, benzoic, malonic, trifluoroacetic, trichloroacetic, naphthalene-2-sulfonic, and other acids. Pharmaceutically acceptable salt forms can include forms wherein the ratio of molecules comprising the salt is not 1:1. For example, the salt may comprise more than one inorganic or organic acid molecule per molecule of base, such as two hydrochloric acid molecules per molecule of conjugate. As another example, the salt may comprise less than one inorganic or organic acid molecule per molecule of base, such as two molecules of conjugate per inorganic or organic acid molecule.

The terms “carrier” and “pharmaceutically acceptable carrier” as used herein refer to a diluent, adjuvant, excipient, or vehicle with which a compound is administered or formulated for administration. Non-limiting examples of such pharmaceutically acceptable carriers include liquids, such as water, saline, and oils; and solids, such as gum acacia, gelatin, starch paste, talc, keratin, colloidal silica, urea, and the like. In addition, auxiliary, stabilizing, thickening, iso osmotic, cryo-preservatives, lubricating, flavoring, and coloring agents may be used. Other examples of suitable pharmaceutical carriers are described in Remington's Science and Practice of Pharmacy (23^{rd} edition, ISBN 9780128200070) and Handbook of Pharmaceutical Excipients (8^{th} edition, 978-0-85-711271-2), each herein incorporated by reference in their entirety.

As used herein, the term “target molecule” refers to any molecule having a therapeutic or diagnostic application or a targeting function, or a vehicle with which a compound is administered or formulated for administration, wherein the target molecule is capable of forming a linkage with an active functional group on a POZ polymer or a POZ derivative of the present disclosure, including, but not limited to, a therapeutic agent (such as but not limited to a drug), a diagnostic agent, a targeting agent, an organic small molecule, an oligonucleotide, a polypeptide, an antibody, an antibody fragment, a protein, a carbohydrate such as heparin or hyaluronic acid, or a lipid such as a glycerolipid, glycolipid, or phospholipid.

As used herein, “lipid” or “lipid portion” means (i) an organic compound that includes an ester of fatty acid or a derivative thereof and is characterized by being insoluble in water, but soluble in many organic solvents, and includes, but is not limited to, simple lipids such as fats, oils, and waxes, compound lipids such as phospholipids, glycolipids, cationic lipids, non-cationic lipids, neutral lipids, and anionic lipids, and derived lipids such as steroids, as well as (ii) an organic compound that does not include an ester of fatty acid, but mimics such an organic compound through its amphipathic character, i.e., it possesses both hydrophobic and hydrophilic portions, and, thus, is able to aggregate in a specific manner to form layers, vesicles and LNPs in aqueous environments.

As used herein, “small interfering RNA (siRNA)” mean a class of double-stranded RNA molecules, 16-40 nucleotides in length, that are involved in the RNA interference (RNAi) pathway, where it interferes with the expression of a specific gene. In addition to their role in the RNAi pathway, siRNAs also act in RNAi-related pathways.

As used herein, “sgRNA” mean a class of guide RNA molecules that are involved in CRISPR-Cas9 genome editing, where the sgRNA provides a template for precise genome editing to minimize “off-target” editing and maximize “on-target” editing.

As used herein, “saRNA” mean a class of RNA molecules that are “self-amplifying” or “self-replicating” due to the nature of the encoded protein, typically a replicase, that result in multiple copies of the RNA.

As used herein, “RNA” means a molecule comprising at least one ribonucleotide residue, including siRNA, antisense RNA, single stranded RNA, microRNA, mRNA, noncoding RNA, self-amplifying RNA, sgRNA, gRNA and multivalent RNA. “Ribonucleotide” means a nucleotide with a hydroxyl group at the 2′ position of a B-D-ribo-furanose moiety, and includes but is not limited to, modified ribonucleotides The terms include double-stranded RNA, single-stranded RNA, isolated RNA such as partially purified RNA, essentially pure RNA, synthetic RNA, recombinantly produced RNA, as well as altered RNA that differs from naturally occurring RNA by the addition, deletion, substitution, and/or alteration of one or more nucleotides.

### LNPs Including POZ-lipid Conjugates

Any of the POZ-lipid conjugates discussed below may be used in preparing a LNP in accordance with the present disclosure. In one embodiment, a LNP may be formed with a POZ-lipid conjugate of the present disclosure and at least one of a cationic or ionizable lipid. For example, a LNP may be formed with a POZ-lipid conjugate and a cationic lipid. In another embodiment, a LNP may be formed with POZ-lipid conjugate and an ionizable lipid. In still another embodiment, a LNP includes a POZ-lipid conjugate, a cationic or ionizable lipid, and other lipid components (lacking a polyoxazoline component) that are capable of forming vesicles and/or liposomes (underivatized lipids). Examples of suitable underivatized lipids include, but are not limited to, helper lipids and lipids to stabilize the composition.

In one aspect, a LNP formed according to the present disclosure includes a POZ-lipid conjugate, a cationic or ionizable lipid, and (i) a helper lipid to provide structural support and facilitate endocytosis, and/or (ii) a sterol lipid for stability.

LNPs formed in accordance with the present disclosure may also include a payload. In this aspect, the payload may be an oligonucleotide, protein, or a combination thereof. For example, LNPs of the present disclosure may include (i) an ionizable lipid; (ii) a helper lipid; (iii) a sterol lipid; (iii) a POZ-lipid of the present disclosure; and (iv) an oligonucleotide. In another specific embodiment (not shown), LNPs of the present disclosure may include a cationic lipid, a helper lipid, a sterol lipid, a POZ-lipid of the present disclosure, and an oligonucleotide. In yet another embodiment, LNPs of the present disclosure may include a cationic or ionizable lipid, a helper lipid, a sterol lipid, a POZ-lipid of the present disclosure, and a protein.

In one embodiment, the oligonucleotide includes DNA, siRNA, self-replicating mRNA, mRNA including modified nucleosides, and mRNA including naturally occurring nucleosides. In one aspect, the oligonucleotide is DNA. In another aspect, the oligonucleotide is siRNA. In still another aspect, the oligonucleotide is self-replicating mRNA, mRNA including modified nucleosides, or mRNA including naturally occurring nucleosides. In still another aspect, the oligonucleotide is a sgRNA used in genome editing.

In one embodiment, when incorporated in a LNP, the POZ-lipid conjugate is present at a mole ratio of about 0.25 to about 5 mole percent in the lipid layer of the LNP, at a mole ratio of about 0.5 to about 3 mole percent in the lipid layer of the LNP, at a mole ratio of about 0.75 to about 2 mole percent in the lipid layer of the LNP, or at a mole ratio of about 0.8 to about 1.5 mole percent in the lipid layer of the LNP. In other embodiments, the POZ-lipid conjugate is present at a mole ratio of about 1 to about 5 mole percent in the lipid layer of the LNP, at a mole ratio of about 1 to about 2.5 mole percent in the lipid layer of the LNP, or at a mole ratio of about 1.5 to about 5 mole percent in the lipid layer of the LNP.

A non-limiting example of a cationic lipid suitable for use in accordance with the present invention is 1,2-dioleoyl-3-trimethylammonium propane (DOTAP). Suitable ionizable lipids include, but are not limited to, MC3 98, Lipid 319, C12-200, 5A2-SC8, 306Oi10, Moderna Lipid 5, Moderna Lipid H, SM-102, Acuitas A9 [59], Arcturus Lipid 2,2 (8,8) 4C CH3, Genevant CL1. In one embodiment, the cationic or ionizable lipids have a pKa, as measured by the TNS dye-binding assay, in the range of 6-7.

Helper lipids refer to amphipathic lipids that have hydrophobic and polar head group moieties, and which can form spontaneously into bilayer vesicles in water, as exemplified by phospholipids, or are stably incorporated into lipid bilayers, with the hydrophobic moiety in contact with the interior, hydrophobic region of the bilayer membrane, and the polar head group moiety oriented toward the exterior, polar surface of the membrane. Such helper lipids typically include one or two hydrophobic acyl hydrocarbon chains or a steroid group and may contain a chemically reactive group, such as an amine, acid, ester, aldehyde or alcohol, at the polar head group. Non-limiting examples include phospholipids, such as phosphatidyl choline (PC), phosphatidyl ethanolamine (PE), phosphatidic acid (PA), phosphatidyl inositol (PI), and sphingomyelin (SM), where the two hydrocarbon chains are typically between about 14-22 carbon atoms in length, and have varying degrees of unsaturation. Other suitable helper lipids include, but are limited to, glycolipids, such as cerebrosides and gangliosides. In one aspect, the helper lipid is at least one of 1,2-dioleoyl-sn-glycero-3-phosphoethanolamine (DOPE), 1,2-distearoyl-sn-glycero-3-phosphocholine (DSPC), and POPE (1-palmitoyl-2-oleoylsn-glycero-3-phosphoethanolamine).

A suitable sterol lipid for use in accordance with the present disclosure is cholesterol. In one particular embodiment, a LNP in accordance with the present disclosure includes a cationic or ionizable lipid combined with: (i) DSPC; (ii) cholesterol; (iii) a POZ-lipid of the present disclosure; and (iv) an oligonucleotide.

It is important to note that changes or additions (even very minor) to the LNPs of the present disclosure may impact not only the structure of the LNP, but also the delivery of the encapsulated payload. For example, when a sterol lipid such as cholesterol is included in a LNP composition along with an ionizable lipid and a POZ-lipid described herein, the resulting LNP has a single bilayer. If phytosterol is added, the structure of the LNP becomes more complex and, thus, may deliver the payload differently. In this vein, compositions of the present disclosure including the POZ-lipid conjugates may be unilamellar or non-unilamellar.

The particle size of LNPs made in accordance with the present disclosure can vary. In one embodiment, LNPs formed in accordance with the present disclosure are amphiphilic spherical vesicles formed by one or more lipid bilayers enveloping an aqueous core with size ranging from about 10 nm to about 10 microns. In another embodiment, a LNP formed in accordance with the present disclosure has a particle size of about 25 nm to about 8 microns. In yet another embodiment, a LNP formed in accordance with the present disclosure has a particle size of about 30 nm to about 5 microns. In this aspect, the particle size of the LNP may be between about 20 nm to about 3 microns. In another embodiment, the LNP may be between about 10 nm and about 1000 nm, between about 25 nm and about 500 nm, between about 35 nm and about 250 nm, between about 40 nm and about 150 nm, or between about 45 nm and about 100 nm. Methods of size fractionation are disclosed herein. However, in certain aspects, size fractionation is not required.

In some aspects, the LNPs formed in accordance with the present disclosure have a particle size that is greater than the particle size of it PEG-LNP counterpart (i.e., a LNP that is identical to a POZ-LNP formed in accordance with the present disclosure except for substitution of PEG for POZ (in the lipid conjugate)) by at least about 25 percent. For example, a POZ-LNP formed in accordance with the present disclosure may have a particle size that is about 25 percent to about 99 percent more than its PEG-LNP counterpart. In one embodiment, a POZ-LNP of the present disclosure has a particle size that is at least 50 percent greater than its PEG-LNP counterpart.

The LNP composition of the present disclosure may be prepared by a variety of methods. In one embodiment, the liposomes are prepared by the reverse-phase evaporation method (Szoka et al. PNAS 1978 vol. 75, 4194-4198; Smirnov et al., Byulleten' Éksperimental'noi Biologii i Meditsiny, 1984, Vol. 98, pp. 249-252; U.S. Pat. No. 4,235,871). In this method, an organic solution of liposome-forming lipids, which may include the polyoxazoline-lipid conjugate, either with or without a linked target molecule, is mixed with a smaller volume of an aqueous medium, and the mixture is dispersed to form a water-in-oil emulsion, preferably using pyrogen-free components. The target molecule to be delivered is added either to the lipid solution, in the case of a lipophilic target molecule, or to the aqueous medium, in the case of a water-soluble target molecule. The lipid solvent is removed by evaporation and the resulting gel is converted to liposomes. The reverse phase evaporation vesicles (REVs) have typical average sizes between about 0.2-0.4 microns and are predominantly oligolamellar, that is, contain one or a few lipid bilayer shells. The REVs may be readily sized, as discussed below, by extrusion to give oligolamellar vesicles having a selected size preferably between about 0.05 to 0.2 microns.

In addition, multilamellar vesicles (ML Vs) can be created. In this method, a mixture of liposome-forming lipids, which may include the polyoxazoline-lipid conjugate, either with or without a linked target molecule, as described herein are dissolved in a suitable solvent is evaporated in a vessel to form a thin film. The thin film is then covered by an aqueous medium. The lipid film hydrates to form MLVs. MLVs generally exhibit sizes between about 0.1 to 10 microns. MLVs may be sized down to a desired size range by extrusion and other method described herein.

One effective sizing method for REVs and MLVs involves extruding an aqueous suspension of the liposomes through a polycarbonate membrane having a selected uniform pore size, typically 0.05, 0.08, 0.1, 0.2, or 0.4 microns (Szoka et al. PNAS 1978 vol. 75, 4194-4198). The pore size of the membrane corresponds roughly to the largest sizes of liposomes produced by extrusion through that membrane, particularly where the preparation is extruded two or more times through the same membrane. Process for sizing MLVs of larger sizes is provided by Zhu et al. (PLoS One. 2009; 4(4): e5009. Epub 2009 April 6).

When small particle sizes are desired, the REV or MLV preparations can be treated to produce small unilamellar vesicles (SUVs) that are characterized by sizes in the 0.04-0.08 micron range. Such particles may be useful in targeting tumor tissue or lung tissue where the particles may be absorbed through capillary walls (larger than 0.1 microns may not be absorbed).

LNPs formed in accordance with the present disclosure may have a polydispersity index (i.e., a ratio of mass average molecular mass to the number average molecular mass) that ranges from about 0.05 to about 0.3. In some aspects, the LNPs formed in accordance with the present disclosure have a polydispersity index that is about 0.08 to about 0.25. In other aspects, the polydispersity index of a POZ-LNP formed in accordance with the present disclosure is about 0.1 to about 0.2. In still other aspects, LNPs formed in accordance with the present disclosure have a polydispersity index that is about 0.0.1 to about 0.18.

In some aspects, the LNPs formed in accordance with the present disclosure have a polydispersity index that is substantially similar as the polydispersity index of a comparable PEG-LNP. In this context, “substantially similar” means that the polydispersity index of POZ-LNP varies from PEG-LNP by no more than about 0.05. For example, a POZ-LNP formed in accordance with the present disclosure may have a polydispersity index that differs from the polydispersity of its PEG-LNP counterpart by no more than about 0.03.

Furthermore, the POZ-lipid conjugate may be introduced into the LNP after the liposomes are formed using the techniques described above. In this approach, the preformed liposomes are incubated in the presence of a POZ-lipid conjugate; the POZ-lipid conjugate is incorporated into the liposome by diffusion. The concentration of the POZ-lipid conjugate free in solution or taken up by the liposome may be monitored and the process terminated when a desired concentration of the POZ-lipid conjugate in the LNP is reached. The incubation solution may contain surfactants or other agents to facilitate diffusion of the POZ-lipid conjugates into the LNP.

The LNP may be treated to remove extraneous components prior to use. For example, if surfactants are used as discussed above, the excess surfactants may be removed prior to use. In addition, where a payload, such as an oligonucleotide discussed above, is entrapped in the LNP composition, excess or non-entrapped payload may be removed prior to use. Separation techniques to accomplish this task are known in the art and the particular method selected may depend on the nature of the component to be removed. Suitable methods include, but are not limited to, centrifugation, dialysis and molecular-sieve chromatography. The composition can be sterilized by filtration through a conventional 0.22 micron depth filter.

LNPs can be prepared by the traditional method that involves the hydration of a lipid film containing POZ-lipid conjugate, an ionizable lipid, helper lipids, and cholesterol. This process involves the dissolution of these materials in organic solvent such as chloroform or dichloromethane and then evaporating the solvent to produce a thin film. The film is then hydrated with an aqueous buffer containing the drug or nucleic acid to passively encapsulate the payload. LNPs of heterogeneous particles with a low encapsulation are normally formed, which requires size reduction by extrusion or sonication.

In some embodiments, formulations may be prepared via microfluidic synthesis. In other embodiments, formulations may be prepared any method suited for LNP production including, but not limited to, T-mixers, hand pipetting, and syringe pumps connected to a PDMS microfluidic chip.

For example, one suitable technique uses rapid mixing with a microfluidizer. Lipid stock solutions are prepared by dissolving the lipids in an organic solvent, such as ethanol. Aqueous stock solutions contain the nucleic acid dissolved in a buffer solution of known pH, ionic strength and buffer capacity. The two stock solutions are passed through a micromixer at a predetermined rate to allow for the cationic lipid to interact with the negatively charged nucleic acid, resulting in higher encapsulation efficiencies (i.e., >80 percent) and homogeneous size distribution. The aqueous-to-organic solvent ratios during the mixing process is important. The organic solvent is removed by dialysis, tangential flow filtration or centrifugation or other technique. LNPs of defined sizes are produced by controlling the microfluidic operating parameters, resulting in LNPs of low polydispersity and uniform particle size. The average particle diameter, polydispersity, and zeta potential of the LNPs are three methods used to characterize the preparation. LNPs formed in accordance with the present disclosure may have an encapsulation efficiency that ranges from about 75 percent to about 100 percent. In some aspects, the LNPs formed in accordance with the present disclosure have an encapsulation efficiency of about 80 to about 99 percent. In other aspects, the encapsulation efficiency of a POZ-LNP formed in accordance with the present disclosure is about 85 to about 95 percent. In some aspects, the LNPs formed in accordance with the present disclosure have an encapsulation efficiency that is substantially similar as the encapsulation efficiency of a comparable PEG-LNP. In this context, “substantially similar” means that the encapsulation efficiency of POZ-LNP varies from PEG-LNP by no more than about 5 percent. For example, a POZ-LNP formed in accordance with the present disclosure may have an encapsulation efficiency that differs from the encapsulation of its PEG-LNP counterpart by no more than about 3 percent.

The ratio of POZ-lipid to ionic lipid to cholesterol can be varied in order to allow for optimal size, high payload release and transfection, and improved stability of the hydrated formulation. In one embodiment, the mol percent of POZ-lipid in the LNP is about 0.5 to 60 percent. In another embodiment, the mol percent of POZ-lipid is about 1 to about 40 percent. In still another embodiment, the POZ-lipid is present in an amount of about less than 10 percent of the total amount of lipids in the LNP. In this aspect, the POZ-lipid may be present in an amount of about 0.5 to about 5 percent, about 1 to about 4 percent, or about 1.5 to about 3.5 percent. In this aspect, the remainder of the LNP may be about 35 to about 50 percent sterol lipid, about 30 percent to about 70 percent cationic lipid, and about 5 percent to about 15 percent helper lipid.

In one embodiment, the LNP includes a lipid bilayer encapsulating an aqueous core where the lipid bilayer includes at least one POZ-lipid conjugate, wherein the average molecular weight of the POZ is between about 0.5 and 5 kDa and the aqueous core includes an oligonucleotide. In another embodiment, the LNP includes a lipid bilayer encapsulating an aqueous core where the lipid bilayer includes at least one POZ-lipid conjugate, wherein the average molecular weight of the POZ is between about 2 and 5 kDa and the aqueous core includes an oligonucleotide.

The oligonucleotide can be encapsulated into the LNP with a high efficiency. In one embodiment, the oligonucleotide is encapsulated into the LNP with an efficiency of at least 75 percent. In another embodiment, the oligonucleotide is encapsulated into the LNP with an efficiency of about 80 to about 99 percent. In still another embodiment, the oligonucleotide is encapsulated into the LNP with an efficiency of about 85 to about 95 percent. In still another embodiment, the oligonucleotide is encapsulated into the LNP with an efficiency of about 90 to about 95 percent. yet another embodiment, the oligonucleotide is encapsulated into the LNP with an efficiency of greater than about 95 percent.

### POZ-Lipid Conjugates

The POZ-lipid conjugates of the present disclosure include a lipid portion linked to a polyoxazoline (POZ) polymer. In this aspect, the lipid portion of the POZ-lipid conjugate includes at least one hydrophobic moiety. In another embodiment, the lipid portion includes two hydrophobic moieties. In this aspect, the hydrophobic moieties may be acyl chains, alkyl chains, or combinations thereof. The acyl and alkyl chains may vary in length. In addition, the acyl and alkyl chains may be saturated or contain one or more areas of unsaturation (such as one or more double bonds).

Regardless of the number of hydrophobic moieties, the lipid portion also includes a chemical group capable of forming a linkage with a chemical group on the POZ polymer. In this aspect, the chemical group may be an amine group, hydroxyl group, aldehyde group, carboxylic acid group, and combinations thereof with other chemical groups not excluded. In one embodiment, the lipid portion may contain a reactive amino group that can be used to form a linkage with the POZ polymer.

In some embodiments, the chemical group on the lipid may be located at the hydrophilic head group position. And, as mentioned above, the POZ polymer may be conjugated to the lipid via an appropriate chemical group on the initiator or the terminal end of the polymer or via an appropriate chemical group at a pendant position on the polymer.

As will be discussed in greater detail below, the nature of the linkage depends on the chemical group present on the POZ polymer and the chemical group present on the lipid portion. In some embodiments, the linkage is degradable in the presence of certain enzymes. In other embodiments, the linkage is stable in the presence of these same enzymes.

In one embodiment, the lipid portion of the POZ-lipid conjugate is a non-charged lipid. For example, any non-charged lipid capable of forming a layer, vesicle and/or LNP composition, either alone or in combination with other lipid components, is suitable for use in forming a POZ-lipid conjugate of the present disclosure. In another embodiment, the lipid portion may be synthetic or naturally occurring.

The lipid portion of the POZ-lipid conjugate may be selected to impart desired characteristics to the LNPs described herein. For example, the degree of unsaturation of the lipid may be selected to provide desired properties to the LNPs described herein. For example, increasing the degree of unsaturation of the lipid portion may impart fluidity to the LNP composition. In addition, a cis configuration around the area of unsaturation may also impart increased fluidity to the LNP composition. Likewise, a saturated lipid portion may impart rigidity to the LNP composition. The fluidity and/or rigidity may be selected to control, at least in part, the stability of the LNP and/or the rate of release of a POZ-lipid conjugate from the LNP composition.

In one embodiment, the lipid portion of the POZ-lipid conjugate is a phospholipid. For example, the lipid portion of the POZ-lipid conjugate may be phosphatidyl glycerol (PG), phosphatidyl choline (PC), phosphatidyl ethanolamine (PE), phosphatidic acid (PA), phosphatidyl inositol (PI), phosphatidylserine (PS), or combinations thereof.

In another embodiment, the lipid portion of the POZ-lipid conjugate is a glycerolipid. For example, the lipid portion may be αβ-diacylglycerol.

In a further embodiment, the lipid portion of the POZ-lipid conjugate is a dialkylamine. For example, the lipid portion may be dimyristylamine.

In a certain aspect, at least one of the two acyl or alkyl chains of the lipid portion in the POZ-lipid conjugate is saturated. In another aspect, each the two acyl or alkyl chains of the lipid in the POZ-lipid conjugate is saturated. In yet another aspect, one of the two acyl or alkyl chains of the lipid in the POZ-lipid conjugate is saturated and the other acyl or alkyl chain is unsaturated. In still another aspect, each the two acyl or alkyl chains of the lipid in the POZ-lipid conjugate is unsaturated. When one or more acyl or alkyl chains of the lipid in the POZ-lipid conjugate are unsaturated, the acyl or alkyl chain may contain from 1 to 6, from 1 to 4, from 1 to 3, or from 1 to 2 areas of unsaturation. The double bond(s), when present, may be in the cis or trans configuration, or a mixture of cis and trans configuration.

In one aspect, at least one of the two acyl or alkyl chains of the lipid in the POZ-lipid conjugate is from 1 to 5 carbons in length, from 6 to 10 carbons in length, from 11 to 16 carbons in length, or from 17-21 carbons in length. In another aspect, each of the two acyl or alkyl chains of the lipid in the POZ-lipid conjugate is from 1 to 5 carbons in length, from 6 to 10 carbons in length, from 11 to 16 carbons in length, or from 17-21 carbons in length. Such acyl or alkyl chains, regardless of the length, include both even and odd chain lengths and may be saturated or unsaturated as described above.

In one particular aspect, at least one of the two acyl or alkyl chains of the lipid portion in the POZ-lipid conjugate is from 6 to 10 carbons in length. In another aspect, each of the two acyl or alkyl chains of the lipid in the POZ-lipid conjugate is from 6 to 10 carbons in length. Such acyl or alkyl chains, regardless of the length, includes both even and odd chain lengths and may be saturated or unsaturated as described above.

In another aspect, at least one of the two acyl or alkyl chains of the lipid portion in the POZ-lipid conjugate is from 11 to 16 carbons in length. In another aspect, each of the two acyl or alkyl chains of the lipid in the POZ-lipid conjugate is from 11 to 16 carbons in length. Such acyl or alkyl chains, regardless of the length, includes both even and odd chain lengths and may be saturated or unsaturated as described above.

In yet another aspect, at least one of the two acyl or alkyl chains of the lipid in the POZ-lipid conjugate is from 17 to 21 carbons in length. In still another aspect, each of the two acyl or alkyl chains of the lipid in the POZ-lipid conjugate is from 17 to 21 carbons in length. Such acyl or alkyl chains, regardless of the length, includes both even and odd chain lengths and may be saturated or unsaturated as described above.

In one aspect, each of the two acyl or alkyl chains of the lipid in the POZ-lipid conjugate is 5, 6, 7, 8, 9, 10, 11, 12, 13, 14, 15, 16, 17, 18, 19, 20, or 21 carbons in length, which acyl or alkyl chains are saturated or unsaturated. In another aspect, each of the two alkyl or acyl chains of the lipid in the POZ-lipid conjugate is an acyl chain of 11, 12, 13, 14, 15, or 16 carbons in length, which acyl chains are saturated or unsaturated. In a further aspect, each of the two acyl or alkyl chains of the lipid in the POZ-lipid conjugate is an acyl chain of 12, 13, 14, or 15 carbons in length, which acyl chains are saturated or unsaturated. In still a further aspect, each of the two acyl or alkyl chains of the lipid in the POZ-lipid conjugate is an acyl chain of 13 or 14 carbons in length, which are acyl chains are saturated or unsaturated. In still another aspect, each of the two alkyl or acyl chains of the lipid in the POZ-lipid conjugate is an alkyl chain of 11, 12, 13, 14, 15, or 16 carbons in length, which alkyl chains are saturated or unsaturated. In a further aspect, each of the two acyl or alkyl chains of the lipid in the POZ-lipid conjugate is an alkyl chain of 12, 13, 14, or 15 carbons in length, which alkyl chains are saturated or unsaturated. In still a further aspect, each of the two acyl or alkyl chains of the lipid in the POZ-lipid conjugate is an alkyl chain of 13 or 14 carbons in length, which are alkyl chains are saturated or unsaturated.

In still another aspect, each acyl or alkyl chain of the lipid portion in the POZ-lipid conjugate has the same length and is unsaturated. For example, the acyl or alkyl chains may be 6, 7, 8, 9, 10, 11, 12, 13, 14, 15, 16, 17, 18, 19, 20, or 21 carbons in length, 11, 12, 13, 14, 15, or 16 carbons in length, 12, 13, 14, or 15 carbons in length, or 13 or 14 carbons in length and unsaturated.

In another aspect, the lipid portion in the POZ-lipid conjugate is 1,2-dimyristoyl-sn-glycerol or 1,2-dilauroyl-sn-glycerol. In still another aspect, the lipid portion in the POZ-lipid conjugate is di(tetradecyl)acetamide or di(dodecyl)acetamide. In yet another aspect, the lipid portion in the POZ-lipid conjugate is N,N-di(tetradecyl)acetamide or N,N-di(dodecyl)acetamide. In still another aspect, the lipid portion may be 1, 2-distearoyl-sn-glycero-3-phosphoethanolamine-poly (ethylene glycol) (DSPE).

A variety of POZ polymers may be used in the POZ-lipid conjugates. The POZ may be a linear POZ polymer, a branched POZ polymer, a pendent POZ polymer or a multi-armed POZ polymer. Representative POZ polymers are described in U.S. Pat. Nos. 7,943,141, 8,088,884, 8,110,651, 8,101,706, 8,883,211, and 9,284,411, and U.S. patent application Ser. Nos. 13/003,306, 13/549,312 and 13/524,994, each of which is incorporated by reference in its entirety for such teachings. The polyoxazoline polymer may be a homopolymer; likewise, the polyoxazoline polymer may be a random or block copolymer containing one or more units of a first polyoxazoline polymer separated by one or more units of a second polyoxazoline polymer. Likewise, the POZ may be a poly(methyloxazoline) (PMOZ), which is quite hydrophilic, or poly(ethyloxazoline) (PEOZ), which is less hydrophilic. For example, in some embodiments, the POZ is PEOZ.

In one embodiment, the POZ polymer is prepared by living cation polymerization. Other methods known in the art may also be used to prepare the POZ polymer. As discussed in more detail below, the POZ can be conjugated directly to the lipid or may be linked to the lipid via a linker moiety. Any linker moiety suitable for coupling the POZ to a lipid can be used including, but not limited to, non-ester-containing linker moieties and ester-containing linker moieties. And, as discussed in more detail below, the POZ polymer may be conjugated to the lipid portion via an appropriate chemical group on the initiator or the terminal end of the polymer or via an appropriate chemical group at a pendant position on the polymer.

In the present disclosure, whenever a polyoxazoline derivative or polyoxazoline polymer is mentioned, the polyoxazoline polymer may be one characterized with low polydispersity (PD) values and/or increased purity, as such polymers are useful in pharmaceutical applications. In a particular embodiment, the methods of the present disclosure provide for polyoxazoline derivatives with low PD values at increased molecular weight (MW) values. In one embodiment, for example, the POZ portion has a molecular weight of about 500 to about 10000 Daltons. In another embodiment, the POZ portion has a molecular weight of about 500 to about 5,000 Daltons. In still another embodiment, the POZ portion has a molecular weight of about 1,000 Daltons about 2,500 Daltons. In yet another embodiment, the POZ portion has a molecular weight of about 2,000 Daltons to about 5,000 Daltons. In still another embodiment, the POZ portion has a molecular weight of about 5,000 Daltons to about 10,000 Daltons. In such embodiments, at least one polyoxazoline polymer chain has a polydispersity value of less than or equal to 1.2, less than or equal to 1.1, or less than or equal to 1.05. Methods of synthesizing polyoxazoline polymers and derivatives thereof with low PD values are discussed in International Application Nos. PCT/US2008/002626 and PCT/US2008/078159, which are incorporated by reference in their entireties for such teaching.

In general, the covalent attachment of POZ to a lipid is accomplished by reaction of an active chemical group on the POZ polymer with a complementary chemical group on the lipid. The chemical groups on the POZ polymer and/or the lipid may be activated prior to the reaction (such as, but not limited to, removal of any protecting groups). A hydroxyl, amine or carboxyl group may be activated for coupling by monofunctional activating agents, such as N-hydroxysuccinimide, ethylchloroformate, DCCD, Woodward's Reagent K, cyanuric acid and trifluoromethanesulfonyl chloride, among others. A number of bifunctional crosslinking reagents containing groups with different reactivities, such as some diisocyanates, may also be used.

In one embodiment, the POZ-lipid conjugate may be represented by the general formula I where the lipid is attached to the polymer chain at the terminating terminus:

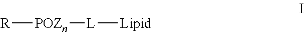

or a pharmaceutically acceptable form thereof, such as, but not limited to, a pharmaceutically acceptable salt, wherein,
R is an initiating group;
POZ is a polyoxazoline polymer;
n is 1 to 1,000 and represents the number of monomer units comprising the polyoxazoline polymer;
L is a linking group optionally containing a cleavable moiety in which the rate of cleavage is controlled and represents a direct linkage through a reactive group on the lipid and a reactive group on the polymer, wherein the direct linkage may form a cleavable moiety in which the rate of cleavage can be controlled from highly labile to stable; and
Lipid represents a lipid moiety as described herein.

In one embodiment of Structure I, the POZ polymer contains at least one reactive group capable of forming a linkage with a Lipid or a linking group. The linkage (whether a direct linkage or a linkage utilizing a linking group) between the polymer and lipid may be formed between any reactive group on the polymer backbone, including a reactive group at the terminal position or a pendent position (at the terminus), and a reactive group on the lipid. In one aspect, the linkage between the linking group and the polymer may be formed at the terminal end of the polymer. In another aspect, the linkage between the linking group and the polymer may be formed at a pendent position on the polymer. Furthermore, the linkage (whether a direct linkage or a linkage utilizing a linking group) may include components of the reactive group that was originally present on the polymer or the lipid. The linkage (whether a direct linkage or a linkage utilizing a linking group) may be physiologically degradable. In this aspect, the linkage may contain a cleavable moiety. Suitable linking groups include, but are not limited to ethers, esters, amines, amides, and combinations thereof.

In one aspect of this embodiment, L is a stable linkage. In another aspect of this embodiment, L is a physiologically degradable and includes a cleavable moiety. For example, in one embodiment, L may be selected from esters, carboxylate esters (—C(O)—O—), carbonate esters (—O—C(O)—O—), carbamates (—O—C(O)—NH—) and amides (—C(O)—NH—). In yet another aspect of this embodiment, L is a linkage that does not contain a cleavable moiety.

Exemplary R groups include, but are not limited to, hydrogen, alkyl and substituted alkyl. In one embodiment, the initiating group is an alkyl group, such as a C1 to C4 alkyl group. In a specific embodiment of the foregoing, the initiating group is a methyl group. In another embodiment, the initiating group is H. In some aspects, the initiating group may be selected to lack an active functional group. In other aspects, the initiating group may be selected to include an active functional group. Additional suitable initiating groups are disclosed in U.S. Pat. Nos. 7,943,141, 8,088,884, 8,110,651, 8,101,706, 8,883,211, and 9,284,411, and U.S. patent application Ser. Nos. 13/003,306, 13/549,312 and 13/524,994, each of which is incorporated by reference in its entirety for such teachings.

In some embodiments, R is H or CH₃.

In one aspect, the POZ polymer in Structure I may be a polymer represented by [N(COR₂)CH₂CH₂]ₙ, wherein R₂ is independently selected for each repeating unit of the POZ polymer from an unsubstituted or substituted alkyl, alkenyl, aralkyl and heterocycylalkyl group, and R is H or CH₃, and the degree of polymerization “n” may range from 15 to 35, 20 to 30, or 25.

In another aspect, the POZ polymer in Structure I may be a polymer represented by [N(COR₂)CH₂CH₂]ₙ, wherein R₂ is independently selected for each repeating unit of the POZ polymer from an unsubstituted and substituted alkyl, and R is H or CH₃, and n may range from 15 to 35, 20 to 30, or 25.

In yet another aspect, the POZ polymer in Structure I is a polymer represented by [N(COR₂)CH₂CH₂]ₙ, wherein R₂ is independently selected for each repeating unit of the POZ polymer from —CH₃ and —CH₂—CH₃, and optionally, R is H or CH₃, and n may range from 15 to 35, 20 to 30, or 25.

When the POZ polymer is a polymer represented by [N(COR₂)CH₂CH₂]ₙ, the POZ polymer of the conjugate is soluble in aqueous environments. The nature of the pendent groups (R₂) can change solubility to some extent. For example, when R₂ is methyl (as in PMOZ) the polymer is highly water soluble, and when R₂ is ethyl (as in PEOZ) the polymer remains water soluble, but to a lesser extent than PMOZ. The solubility of the POZ polymer permits the POZ polymer to extend beyond the liposomal surface and into the extra-liposomal environment. In such a manner the POZ polymer can effectively shield the liposomal surface.

Specific embodiments of the foregoing Structure I include, but are not limited to, L being an amidase-cleavable amide as shown below in I(a)(1) and I(a)(2):

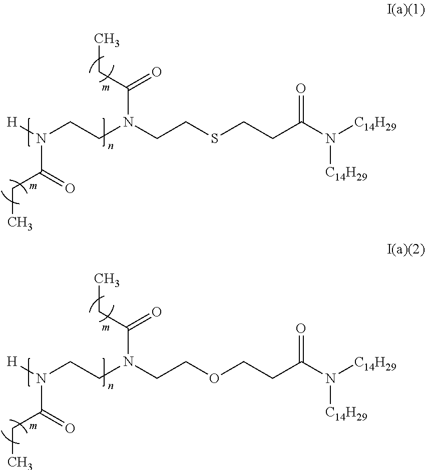

In an alternate embodiment of Structure I, the same lipid group can be incorporated as a stable amine (rather than an amide) as shown below in I(b):

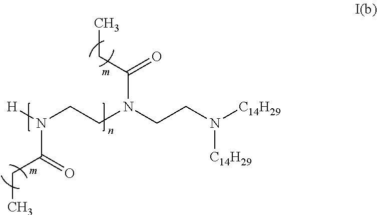

In yet another embodiment of structure I a similar lipid can be coupled via a relatively labile ester linkage as shown below in I(c) and I(d):

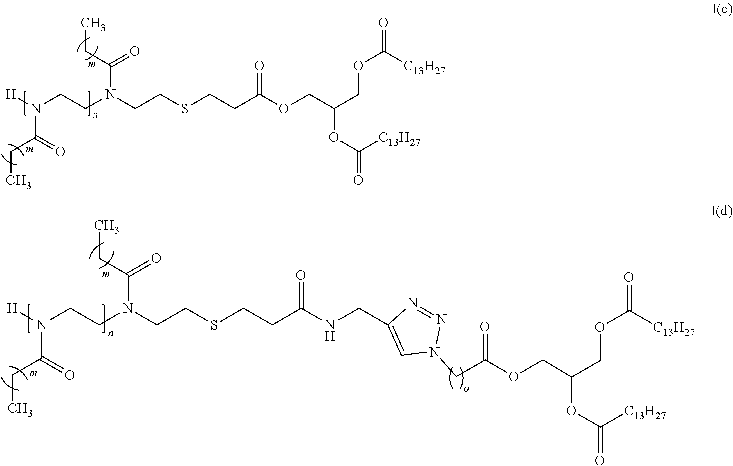

In yet another embodiment of Structure I, a lipid can be coupled via a relatively stable ether linkage as shown below in I(e) and I(f):

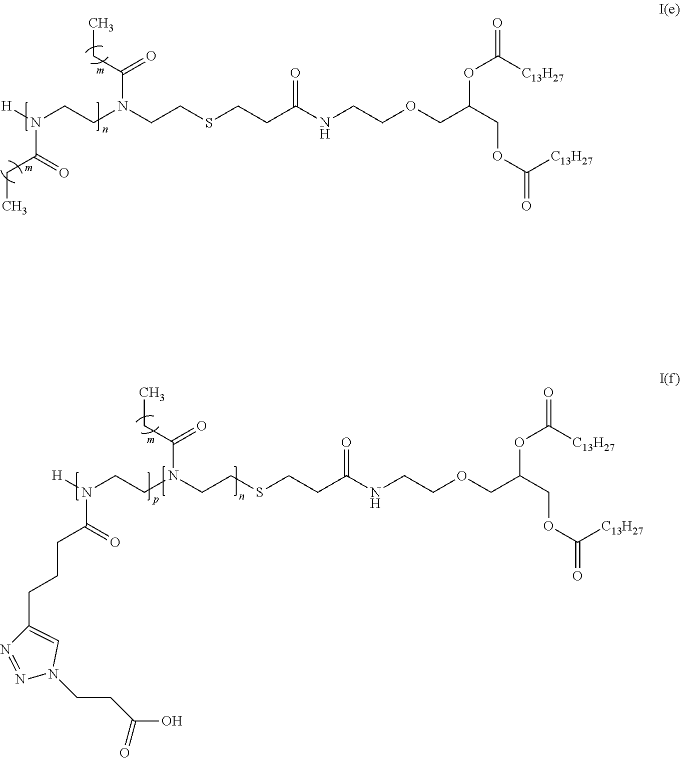

In one embodiment, m is 1 or 2, n ranges from 1 to 1000, o ranges from 1 to 5, and p ranges from 1 to 10 for the Structures I(a)-I(f) (as applicable).

As demonstrated below in the Examples section, the above embodiments of Structure I (I(a)-I(f)) hydrolyze at different rates in plasma, thus illustrating one of the key elements of the novel POZ-lipid conjugates of the present disclosure.

Other specific embodiments of the foregoing Structure I include, but are not limited to, those shown below in I(g) and I(h):

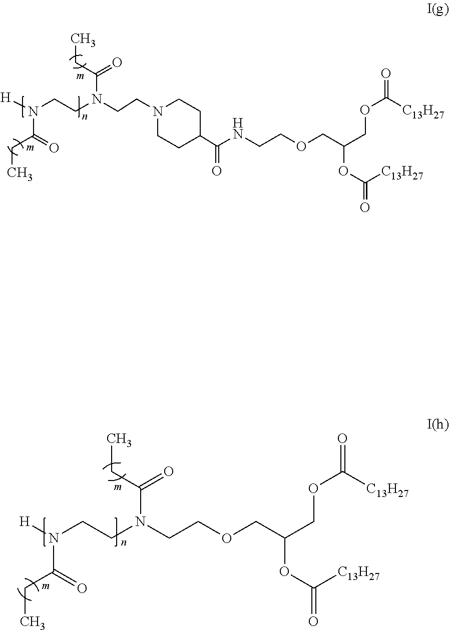

In one embodiment, m is 1 or 2, n ranges from 1 to 1000 for the Structures I(g)-I(h).

In another embodiment of the invention, the POZ-Lipid conjugate may be represented by the general formula II in which the lipid is attached to the polymer chain at the initiator terminus, rather than the terminating terminus as in formula I:

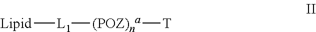

or a pharmaceutically acceptable form thereof, such as, but not limited to, a pharmaceutically acceptable salt, wherein:
POZ is a polyoxazoline polymer of the structure [N(COR₂)CH₂CH₂];
L₁ is a linking group optionally containing a cleavable moiety in which the rate of cleavage can be controlled or represents a direct linkage through a reactive group on the lipid and a reactive group on the polymer, wherein the direct linkage may form a cleavable moiety in which the rate of cleavage can be controlled;
R₂ is independently selected for each repeating unit of the polyoxazoline polymer from an unsubstituted or substituted alkyl, alkenyl, alkyne-substituted alkyl, aralkyl, heterocyclylalkyl, or active functional group;
T is a group at the terminating terminus;
a is ran, which indicates a random copolymer, or block, which indicates a block copolymer; and n is an integer from 1 to 1,000.

The nature of the pendent groups (R₂) can change solubility to some extent. The solubility of the POZ polymer permits the POZ polymer to extend beyond the liposomal surface and into the extra-liposomal environment. In such a manner, the POZ polymer can effectively shield the liposomal surface and prevent aggregation during LNP formation. In addition, without being bound by any particular theory, it is believed that the POZ-lipid conjugate must be “shed” from the LNP surface after administration in order to efficiently deliver the nucleic acid payload. In certain aspects, addition of highly hydrophilic groups to the POZ-lipid conjugate at the R₂ position allows for additional shielding during LNP formation and/or administration and/or enhanced shedding of the POZ-lipid conjugate from the LNP to facilitate delivery of the payload. In one embodiment, R₂ includes at least one hydrophilic group. In another embodiment, R₂ includes a plurality of hydrophilic groups.

L₁ (whether a direct linkage or a linking group) may include components of the reactive group that was originally present on the polymer or the lipid. Suitable linking groups are described herein. L₁ may optionally contain a cleavable moiety, such as, but not limited to, esters, carboxylate esters (—C(O)—O—), carbonate esters (—O—C(O)—O—), carbamates (—O—C(O)—NH—) and amides (—C(O)—NH—).

Exemplary active functional groups include, but are not limited to, alkyne, alkene, amine, oxyamine, aldehyde, ketone, acetal, thiol, ketal, maleimide, ester, carboxylic acid, activated carboxylic acid (such as, but not limited to, N-hydroxysuccinimidyl (NHS) and 1-benzotriazine active ester), an active carbonate, a chloroformate, alcohol, azide, vinyl sulfone, or orthopyridyl disulfide (OPSS). In certain aspects, the active functional group is a hydrophilic group. In certain embodiments, the active functional group is used to add a hydrophilic group to the POZ-lipid conjugate, such as, for example, through click chemistry using an alkyne or an azide active functional group.

In one aspect of this embodiment, L₁ is a stable linkage. In another aspect of this embodiment, L₁ is physiologically degradable and includes a cleavable moiety. In an alternate aspect of this embodiment, L₁ is a linkage that does not contain a cleavable moiety. Suitable L₁ linkages are described herein.

In one aspect of this embodiment, L₁ is a triazole linking group as discussed in more detail below.

In another aspect of this embodiment, T is a terminating nucleophile. For example, T may be Z-B-Q, wherein Z is S, O, or N; B is an optional linking group; and Q is a terminating nucleophile or a terminating portion of a nucleophile.

B groups may include, but are not limited to, alkylene groups. In a particular embodiment, B is —(CH₂)_{y}— where y is an integer selected from 1 to 16.

In a particular aspect, Z is S. POZ-lipid conjugates containing a sulfur group as described herein may be prepared by terminating the POZ cation with a mercaptide reagent, such as, but not limited to, a mercapto-ester (for example, —S—CH₂CH₂—CO₂CH₃) or mercapto-protected amine (for example, —S—CH₂CH₂—NH-tBoc). Such POZ conjugates provide for effective, large-scale purification by ion-exchange chromatography (to remove secondary amines), as well as allowing for control of polydispersity values (with polydispersity values of 1.10 or less) and for the creating of conjugates with higher molecular weight POZ polymers. In another aspect, Z is N. In a further aspect, Z is O.

In certain aspects, Q is inert (i.e., does not contain a functional group). When Q is an inert group, any inert group may be used, including, but not limited to —C₆H₅, alkyl, and aryl mercaptide groups. In an alternate aspect, Q is or contains an active functional group. When Q is or contains an active functional group, suitable functional groups include, but are not limited to, alkyne, alkene, amine, oxyamine, aldehyde, ketone, acetal, thiol, ketal, maleimide, ester, carboxylic acid, activated carboxylic acid (such as, but not limited to, N-hydroxysuccinimidyl (NHS) and 1-benzotriazine active ester), an active carbonate, a chloroformate, alcohol, azide, vinyl sulfone, or orthopyridyl disulfide (OPSS). when Q is or contains an active functional group, Q may be the same as R₂ or Q may be different from R₂ (i.e., Q and R₂ may be chemically orthogonal to one another).

In one particular aspect of this embodiment, R₂ is independently selected for each repeating unit of the polyoxazoline polymer from an unsubstituted or substituted alkyl, and optionally R is H or CH₃, and n is 15 to 35, 20 to 30, 22 to 28, or 25. In another aspect of this embodiment, R₂ is independently selected for each repeating unit of the polyoxazoline polymer from CH₃ and CH₂—CH₃, and optionally R is H or CH₃, and n is 15 to 35, 20 to 30, 22 to 28, or 25. In any of the foregoing aspects, T is Z-B-Q, wherein Z is S, B is —(CH₂)_{y}—, and Q is an inert group, such as, but not limited to, to —C₆H₅, alkyl, and an aryl mercaptide group. Alternatively, in any of the foregoing aspects, T is Z-B-Q, wherein Z is S, B is —(CH₂)_{y}—, and Q is or contains a functional group, such as, but not limited to, alkyne, alkene, amine, oxyamine, aldehyde, ketone, acetal, thiol, ketal, maleimide, ester, carboxylic acid, activated carboxylic acid (such as, but not limited to, N-hydroxysuccinimidyl (NHS) and 1-benzotriazine active ester), an active carbonate, a chloroformate, alcohol, azide, vinyl sulfone, or orthopyridyl disulfide (OPSS).

A specific embodiment of the foregoing Structure II includes, but is not limited to, the following structure II(a) in which the lipid is attached at the initiator terminus while the terminating nucleophile is sulfur:

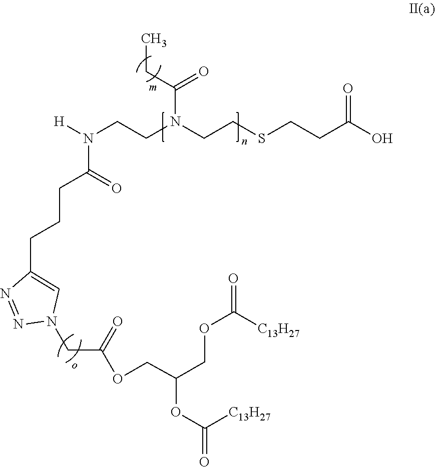

In an alternate embodiment of Structure II as shown below in II(b), the lipid is attached to the initiator terminus while the terminating nucleophile is —OH:

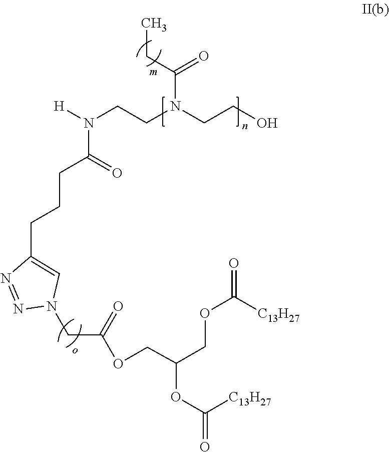

In an alternate embodiment of Structure II shown below in II(c), the lipid is attached to the initiator terminus while the terminating nucleophile is nitrogen, and the lipid is attached via a 2-propionate ester:

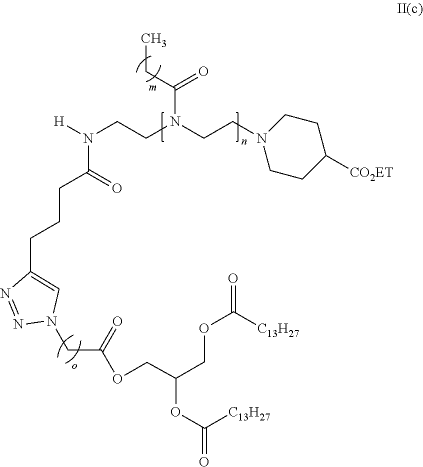

Compounds II(a)-II(c) are made by a “click” reaction of an azide to a pendent alkyne group as shown below:

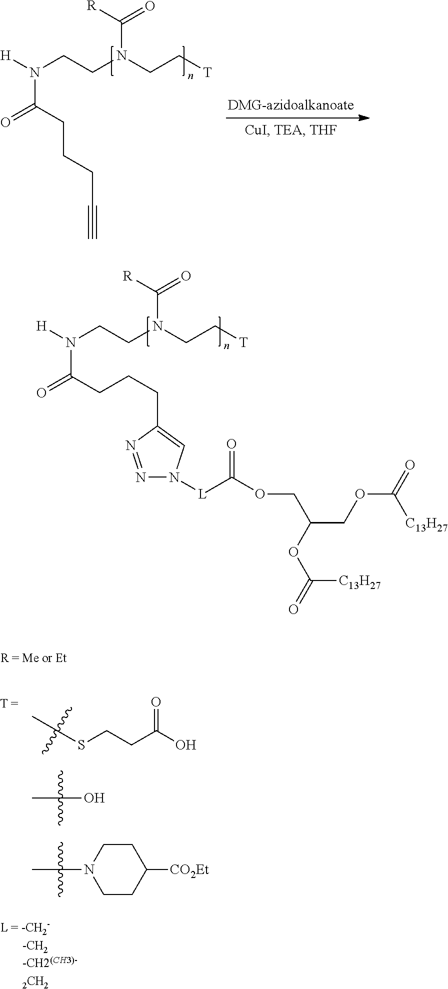

In yet another alternate embodiment of Structure II shown below in II(d), the lipid is attached to the initiator terminus while the terminating nucleophile is sulfur, and the lipid is attached by an acetate ester:

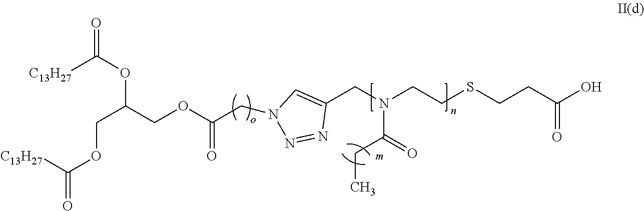

II(d) is made by a “click” reaction of an azide to an alkyne-initiating group as shown below:

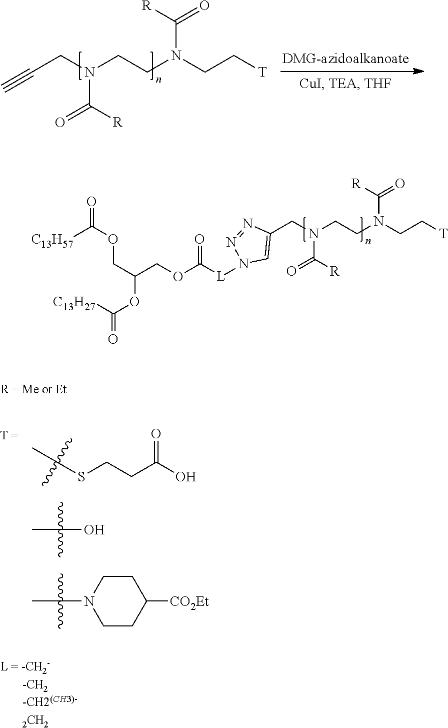

In one embodiment, m is 1-2, n ranges from 1 to 1000, and o ranges from 1 to 5 (as applicable) for Structures II(a)-II(d).

In another embodiment, the POZ-Lipid conjugate may be represented by the general formula III:

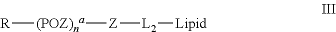

or a pharmaceutically acceptable form thereof, such as, but not limited to, a pharmaceutically acceptable salt, wherein:
POZ is a polyoxazoline polymer of the structure [N(COR₂)CH₂CH₂];
L₂ is a linking group optionally containing a cleavable moiety in which the rate of cleavage can be controlled or represents a direct linkage through a reactive group on the lipid and a reactive group on the polymer, wherein the direct linkage may form a cleavable moiety in which the rate of cleavage can be controlled;
R is an initiating group;
R₂ is independently selected for each repeating unit of the polyoxazoline polymer from an unsubstituted or substituted alkyl, alkenyl, alkyne-substituted alkyl, aralkyl, heterocycylalkyl group, or an active functional group;
Z is S, O, or N;
a is ran, which indicates a random copolymer, or block, which indicates a block copolymer; and
n is an integer from 1 to 1,000.

As with Structure II, the nature of the pendent groups (R₂) can change solubility to some extent. The solubility of the polyoxazoline polymer permits the polyoxazoline polymer to extend beyond the liposomal surface and into the extra-liposomal environment. In such a manner the polyoxazoline polymer can effectively shield the liposomal surface. In addition, the POZ-lipid conjugate must be “shed” from the LNP surface after administration in order to efficiently deliver the nucleic acid payload. In certain aspects, addition of highly hydrophilic groups to the POZ-lipid conjugate at the R₂ position allows for additional shielding during LNP formation and/or administration and/or enhanced shedding of the POZ-lipid conjugate from the LNP to facilitate delivery of the payload.

L₂ (whether a direct linkage or a linking group) may include components of the reactive group that was originally present on the polymer or the lipid. Suitable linking groups are described herein. L₂ may optionally contain a cleavable moiety, such as, but not limited to, esters, carboxylate esters (—C(O)—O—), carbonate esters (—O—C(O)—O—), carbamates (—O—C(O)—NH—) and amides (—C(O)—NH—).

R groups include, but are not limited to, hydrogen, alkyl and substituted alkyl. In one embodiment, the initiating group is an alkyl group, such as a C1 to C4 alkyl group. In a specific embodiment of the foregoing, the initiating group is a methyl group. In another embodiment, the initiating group is H. The initiating group may be selected to lack an active functional group. Alternatively, the initiating group may be selected to include an active functional group. Additional exemplary initiating groups are disclosed in U.S. Pat. Nos. 7,943,141, 8,088,884, 8,110,651, 8,101,706, 8,883,211, and 9,284,411, and U.S. patent application Ser. Nos. 13/003,306, 13/549,312 and 13/524,994, each of which is incorporated by reference in its entirety for such teachings.

Active functional groups include, but are not limited to, alkyne, alkene, amine, oxyamine, aldehyde, ketone, acetal, thiol, ketal, maleimide, ester, carboxylic acid, activated carboxylic acid (such as, but not limited to, N-hydroxysuccinimidyl (NHS) and 1-benzotriazine active ester), an active carbonate, a chloroformate, alcohol, azide, vinyl sulfone, or orthopyridyl disulfide (OPSS). In certain aspects, the active functional group is a hydrophilic group. In certain embodiments, the active functional group is used to add a hydrophilic group to the POZ-lipid conjugate, such as, for example, through click chemistry using an alkyne or an azide active functional group.

In one aspect of this embodiment, L₂ is a stable linkage. In another aspect of this embodiment, L₂ is physiologically degradable and includes a cleavable moiety. In an alternate aspect of this embodiment, L₂ is a linkage that does not contain a cleavable moiety. Suitable L₂ linkages are described herein.

In a particular aspect, Z is S. POZ conjugates containing a sulfur group as described herein may be prepared by terminating the POZ cation with a mercaptide reagent, such as, but not limited to, a mercapto-ester (for example, —S—CH₂CH₂—CO₂CH₃) or mercapto-protected amine (for example, —S—CH₂CH₂—NH-tBoc). Such POZ conjugates provide for effective, large-scale purification by ion-exchange chromatography (to remove secondary amines), as well as allowing for control of polydispersity values (with polydispersity values of 1.10 or less) and for the creating of conjugates with higher molecular weight POZ polymers. In another aspect, Z is N. In a further aspect, Z is O.

In an aspect of this embodiment, R₂ is independently selected for each repeating unit of the polyoxazoline polymer from an unsubstituted or substituted alkyl, and optionally R is H or CH₃, and n is n is 15 to 35, 20 to 30, or 25.

In another aspect of this embodiment, R₂ is independently selected for each repeating unit of the polyoxazoline polymer from CH₃ and CH₂—CH₃, and optionally R is H or CH₃, and n is 15 to 35, 20 to 30, or 25.

In another embodiment, the POZ-Lipid conjugate may be represented by the general formula IV:

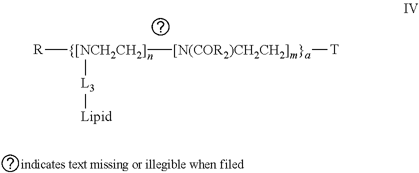

or a pharmaceutically acceptable form thereof, such as, but not limited to, a pharmaceutically acceptable salt, wherein:
R is an initiating group;
R₂ is independently selected for each repeating unit of the polyoxazoline polymer from an unsubstituted or substituted alkyl, alkenyl, alkyne-substituted alkyl, aralkyl, heterocycylalkyl group, or an active functional group;
L₃ is a linking group optionally containing a cleavable moiety in which the rate of cleavage can be controlled or represents a direct linkage through a reactive group on the lipid and a reactive group on the polymer, wherein the direct linkage may form a cleavable moiety in which the rate of cleavage can be controlled;
Lipid is a lipid;
T is a group at the terminating terminus;
a is ran, which indicates a random copolymer, or block, which indicates a block copolymer;
m is an integer from 1 to 100; and
n is an integer from 1 to 5.

In one aspect, n is 1 and this monomer unit is the initial unit adjacent to R.

The nature of the pendent groups (R₂) can change solubility to some extent. The solubility of the polyoxazoline polymer permits the polyoxazoline polymer to extend beyond the liposomal surface and into the extra-liposomal environment. In such a manner the polyoxazoline polymer can effectively shield the liposomal surface. In addition, the POZ-lipid conjugate must be “shed” from the LNP surface after administration in order to efficiently deliver the nucleic acid payload. In certain aspects, addition of highly hydrophilic groups to the POZ-lipid conjugate at the R₂ position allows for additional shielding during LNP formation and/or administration and/or enhanced shedding of the POZ-lipid conjugate from the LNP to facilitate delivery of the payload.

L₃ (whether a direct linkage or a linking group) may include components of the reactive group that was originally present on the polymer or the lipid. Suitable linking groups are described herein. L₃ may optionally contain a cleavable moiety, such as, but not limited to, esters, carboxylate esters (—C(O)—O—), carbonate esters (—O—C(O)—O—), carbamates (—O—C(O)—NH—) and amides (—C(O)—NH—).

R groups include, but are not limited to, hydrogen, alkyl and substituted alkyl. In one embodiment, the initiating group is an alkyl group, such as a C1 to C4 alkyl group. In a specific embodiment of the foregoing, the initiating group is a methyl group. In another embodiment, the initiating group is H. The initiating group may be selected to lack an active functional group. Alternatively, the initiating group may be selected to include an active functional group. Additional exemplary initiating groups are disclosed in U.S. Pat. Nos. 7,943,141, 8,088,884, 8,110,651, 8,101,706, 8,883,211, and 9,284,411, and U.S. patent application Ser. Nos. 13/003,306, 13/549,312 and 13/524,994, each of which is incorporated by reference in its entirety for such teachings.

Active functional groups include, but are not limited to, alkyne, alkene, amine, oxyamine, aldehyde, ketone, acetal, thiol, ketal, maleimide, ester, carboxylic acid, activated carboxylic acid (such as, but not limited to, N-hydroxysuccinimidyl (NHS) and 1-benzotriazine active ester), an active carbonate, a chloroformate, alcohol, azide, vinyl sulfone, or orthopyridyl disulfide (OPSS). In certain aspects, the active functional group is a hydrophilic group. In certain embodiments, the active functional group is used to add a hydrophilic group to the POZ-lipid conjugate, such as, for example, through click chemistry using an alkyne or an azide active functional group.

In one aspect of this embodiment, L₃ is a stable linkage. In another aspect of this embodiment, L₃ is physiologically degradable and includes a cleavable moiety. In an alternate aspect of this embodiment, L₃ is a linkage that does not contain a cleavable moiety. Suitable L₃ linkages are described herein.

In one aspect of this embodiment, T is a terminating nucleophile. In one aspect of this embodiment, T is Z-B-Q, wherein Z is S, O, or N; B is an optional linking group; and Q is a terminating nucleophile or a terminating portion of a nucleophile.

B groups include, but are not limited to, alkylene groups. In a particular embodiment, B is —(CH₂)_{y}- where y is an integer selected from 1 to 16.

In a particular aspect, Z is S. POZ conjugates containing a sulfur group as described herein may be prepared by terminating the POZ cation with a mercaptide reagent, such as, but not limited to, a mercapto-ester (for example, —S—CH₂CH₂—CO₂CH₃) or mercapto-protected amine (for example, —S—CH₂CH₂—NH-tBoc). Such POZ conjugates provide for effective, large-scale purification by ion-exchange chromatography (to remove secondary amines), as well as allowing for control of polydispersity values (with polydispersity values of 1.10 or less) and for the creating of conjugates with higher molecular weight POZ polymers. In another aspect, Z is N. In a further aspect, Z is O.

In certain aspects, Q is inert (i.e., does not contain a functional group). When Q is an inert group, any inert group may be used, including, but not limited to —C₆H₅, alkyl, and aryl mercaptide groups. In other aspects, Q is or contains an active functional group. When Q is or contains an active functional group, exemplary groups include, but are not limited to, alkyne, alkene, amine, oxyamine, aldehyde, ketone, acetal, thiol, ketal, maleimide, ester, carboxylic acid, activated carboxylic acid (such as, but not limited to, N-hydroxysuccinimidyl (NHS) and 1-benzotriazine active ester), an active carbonate, a chloroformate, alcohol, azide, vinyl sulfone, or orthopyridyl disulfide (OPSS). When Q is or contains an active functional group, Q may be the same as R₂ or Q may be different from R₂ (i.e., Q and R₂ are chemically orthogonal to one another).

In an aspect of this embodiment, R₂ is independently selected for each repeating unit of the polyoxazoline polymer from an unsubstituted or substituted alkyl, and optionally R is H or CH₃, m is 15 to 35, 20 to 30, or 25, and n is 1. In another aspect of this embodiment, R₂ is independently selected for each repeating unit of the polyoxazoline polymer from CH₃ and CH₂—CH₃, and optionally R is H or CH₃, m is 15 to 35, 20 to 30, or 25, and n is 1. In any of these aspects, T is Z-B-Q, wherein Z is S, B is —(CH₂)_{y}—, and Q is an inert groups, such as, but not limited to, to —C₆H₅, alkyl, and aryl mercaptide groups. In any of these aspects, T is Z-B-Q, wherein Z is S, B is —(CH₂)_{y}—, and Q is or contains a functional group, such as, but not limited to, alkyne, alkene, amine, oxyamine, aldehyde, ketone, acetal, thiol, ketal, maleimide, ester, carboxylic acid, activated carboxylic acid (such as, but not limited to, N-hydroxysuccinimidyl (NHS) and 1-benzotriazine active ester), an active carbonate, a chloroformate, alcohol, azide, vinyl sulfone, or orthopyridyl disulfide (OPSS).

In a specific embodiment of general formula IV, the POZ-lipid conjugate may be the following:

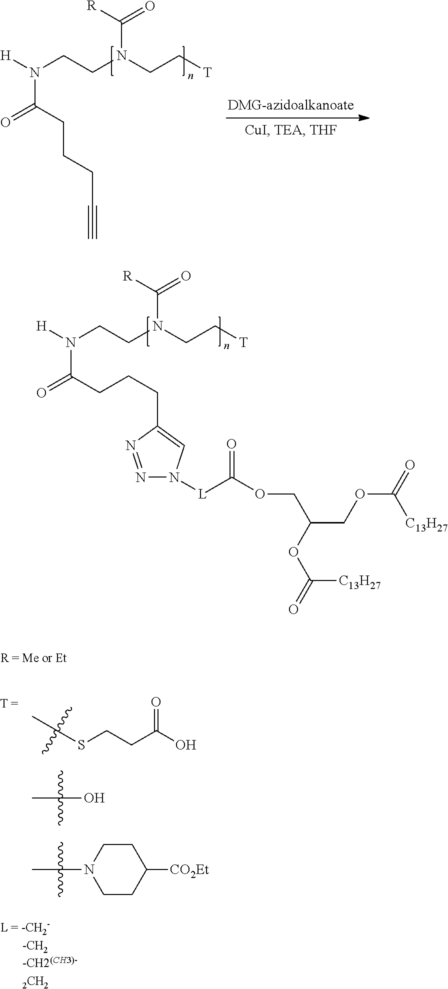

A similar group of compounds is made by coupling an azide to an initiating alkyne group as shown below:

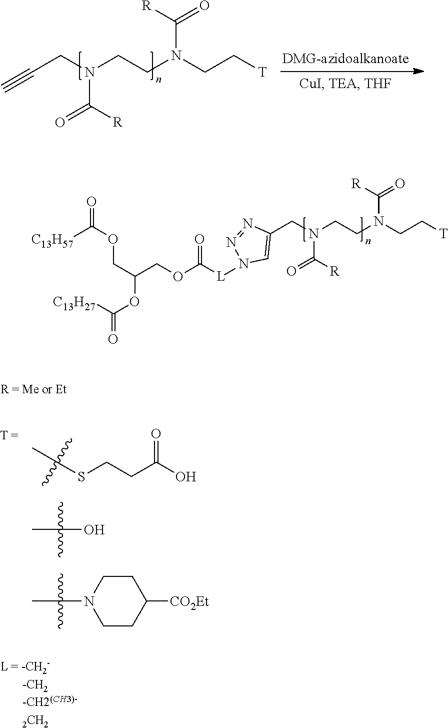

These compounds include a triazole ring. There is evidence that this triazole ring is a privileged scaffold capable of transporting ligands attached to it through the 26S protease, an enzyme responsible for cleavage of polypeptides intracellularly. These so-called proteolysis targeted chimeras (PROTACS) are a promising class of drugs that have been shown to “knockdown” the levels of proteins that bind to one end of the PROTAC and are shuttled through the 26S protease for cleavage. Without being bound to any particular theory, it is contemplated that POZ polymers that incorporate a triazole ring may shuttle POZ polymers through the 26S protease, thus preventing immune presentation of the POZ polymer that does not undergo proteolytic cleavage. Notably, hydrolysis of the degradable ester linkages to release the lipid will leave pendent acid groups attached to the polymer. The inventors have found that soluble POZ with these remaining pendent groups attached via a triazole ring are non-immunogenic, despite being taken up by dendritic cells in the subcutaneous compartment. In addition, labelling studies of a C¹⁴ labelled 20 kD POZ polymer with rotigotine attached showed that the polymer conjugate was taken up via the lymphatics that drain the subcutaneous injection site. The labelled conjugate eventually appears in the spleen where it is almost selectively taken up by the red pulp (the macrophage compartment). Without being bound by any particular theory, it is contemplated that a POZ-lipid LNP of the present disclosure may also be selectively taken up by dendritic cells. In this same vein, without being bound by any particular theory, it is contemplated that a POZ-lipid LNP of the present disclosure may be selectively taken up by macrophages. If so, then the oligonucleotide payload in the LNP may be expressed preferentially in the dendritic cell/macrophage compartments, which could have implications for immune presentation. It is worthwhile to note that, in one aspect, additional triazole-pendent acids may be directly attached as R groups in the above structures. Without being bound by any particular theory, these groups may further contribute to the reduction of immunogenicity of POZ-lipids contained in LNPs.

Additional POZ-lipid conjugates are represented below in formulas V to VII, wherein the POZ polymer is linked to the lipid by L, L₁, L₂, or L₃:

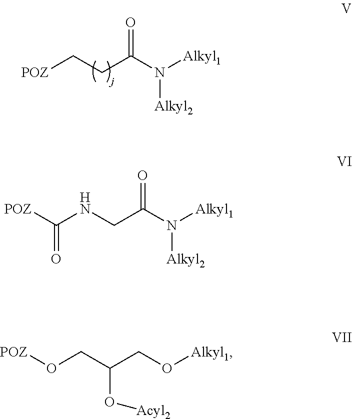

wherein
Alkyl₁ and Alkyl₂ are each independently a saturated or unsaturated alkyl chain of 5, 6, 7, 8, 9, 10, 11, 12, 13, 14, 15, 16, 17, 18, 19, 20, or 21 carbons in length, 11, 12, 13, 14, 15, 16 carbons in length, 12, 13, 14, 15 carbons in length, or 13 or 14 carbons in length; preferably Alkyl₁ and Alkyl₂ have an equal number of carbon atoms and are each unsaturated;
Acyl₁ and Acyl₂ are each independently a saturated or unsaturated acyl chain of 5, 6, 7, 8, 9, 10, 11, 12, 13, 14, 15, 16, 17, 18, 19, 20, or 21 carbons in length, 11, 12, 13, 14, 15, 16 carbons in length, 12, 13, 14, 15 carbons in length, or 13 or 14 carbons in length; preferably Acyl₁ and Acyl₂ have an equal number of carbon atoms and are each unsaturated; and
j is an integer from 1 to 8.

As discussed above, in some of the embodiments described above, the lipid may be linked to the POZ polymer via a cleavable linkage. In one aspect, a linking group is provided between the POZ polymer and the lipid containing a cleavable moiety. In other words, the linking group contains a linkage that is physiologically degradable in that it can be cleaved in specific environments. For example, the linkage may be cleaved in vivo in a subject after administration of a LNP containing a POZ-lipid conjugate of the present disclosure to the subject.

In one embodiment, the cleavable moiety is cleaved by a chemical reaction. In an aspect of this embodiment, the cleavage is by reduction of an easily reduced group, such as, but not limited to, a disulfide. In another embodiment, the cleavable moiety is cleaved by a substance that is naturally present or induced to be present in the subject. In an aspect of this embodiment, such a substance is an enzyme or polypeptide. Therefore, in one embodiment, the cleavable moiety is cleaved by an enzymatic reaction. In yet another embodiment, the cleavable moiety is cleaved by a combination of the foregoing. The linking group may contain portions of the POZ polymer and/or portions of the lipid as such portions have reacted to form the linking group as discussed below.

In this aspect, suitable cleavable moieties include, but are not limited to, esters, carboxylate esters (—C(O)—O—), carbonate esters (—O—C(O)—O—), carbamates (—O—C(O)—NH—) and amides (—C(O)—NH—, including an amide group in a peptide); other releasable moieties are discussed herein. In a particular embodiment, the cleavable moiety is an ester. In another particular embodiment, the cleavable moiety is a carbonate ester or a carboxylate ester. In addition, the linking group may be a naturally occurring amino acid, a non-naturally occurring amino acid or a polymer containing one or more naturally occurring and/or non-naturally occurring amino acids. The linking group may include certain groups from the polymer chain and/or the lipid.

In certain aspects of the POZ-lipid conjugates of formulas I to IV, L, L₁, L₂, and L₃ are physiologically degradable linkages that contain a cleavable moiety independently selected for each occurrence from esters, carboxylate esters (—C(O)—O—), carbonate esters (—O—C(O)—O—), carbamates (—O—C(O)—NH—), amides (—C(O)—NH—), and combinations thereof.

In other certain aspects of the POZ-lipid conjugates of formulas I to IV, L, L₁, L₂, and L₃ are independently selected for each occurrence from —(CH₂)_{f}-(cleavable moiety)-(CH₂)_{g}—, wherein f and g are each an integer independently selected from 0-10, 1-9, 1-8, 1-7, 1-6, or 1-5. In one aspect, f and g are each an integer independently selected from 1-4. In another aspect, any of L, L₁, L₂, and L₃ may be a di-substituted triazole as described herein.

In certain aspects of the POZ-lipid conjugates of formulas I to IV, L, L₁, L₂, and L₃ are independently selected for each occurrence from —(CH₂)_{f}—C(O)—(CH₂)_{g}—, —(CH₂)_{f}—C(O)—(CH₂)ₕ—NHC(O)—(CH₂)_{g}—, —(CH₂)_{f}—NH(CO)—(CH₂)_{g}—C(O)—(CH₂)_{g}—, —(CH₂)_{g}—NHC(O)—(CH₂)_{f}—, —(CH₂)_{f}—C(O)NH—(CH₂)_{g}—, —(CH₂)_{f}—NHOC(O)—(CH₂)_{g}—, —(CH₂)_{f}—OC(O)NH—(CH₂)_{g}—, —(CH₂)_{f}—OC(O)ONH—(CH₂)_{g}—, —(CH₂)_{f}—NHOC(O)O—(CH₂)_{g}—, O—(CH₂)ₕ, (CH₂)ₕ—O or (CH₂)ₕ wherein f, g and h are each an integer independently selected from 0-10, 0-9, 0-8, 0-7, 0-6, or 0-5. In one aspect of this particular embodiment, f, g, and h each may be 0-4.

In another aspect, of the POZ-lipid conjugates of formulas I to IV, L, L₁, L₂, and L₃ are independently selected for each occurrence from a di-substituted triazole that contains a cleavable moiety in one of the R₃ or R₄ groups. The cleavable moiety is preferably present in the R₄ group. In a specific aspect, the di-substituted triazole has the structure:

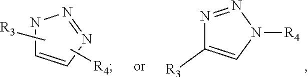

where:

R₃ is a linker linking the triazole moiety to the polymer chain. R₃ may be defined in part by the functional group on the polymer chain; in other words, R₃ may contain a part of the functional group on the polymer chain. In one aspect, R₃ is —C(O)—R₅—, where R₅ is absent or is a substituted or unsubstituted alkyl from 1 to 10 carbons in length.

R₄ is a linker linking the triazole moiety to the lipid. R₄ may be defined in part by the functional group on the agent; in other words, R₄ may contain a part of the functional group on the lipid. In one aspect, R₄ is —R₆-R₇-R₈—, where R₆ is a substituted or unsubstituted alkyl, substituted or unsubstituted aralkyl, R₇ is a group containing the cleavable moiety or a portion of cleavable moiety, and R₈ is absent or O, S, CR_{c}, or NR_{c}, where R_{c} is H or substituted or unsubstituted alkyl. In certain aspects, R₇ and R₈ may combine to form the cleavable moiety. In one embodiment, R₇ is —Rₐ—(O)—R_{b}—, —Rₐ—O—C(O)—R_{b}—, —Rₐ—C(O)—NH-cyclic-O—C(O)—R_{b}— (where cyclic represents substituted or unsubstituted aryl, heterocycloalkyl, heterocycle or cycloalkyl), —Rₐ—C(O)—NH—(C₆H₄)—O—C(O)—R_{b}—, —Rₐ—C(O)—R_{b}—, —Rₐ—C(O)—O—R_{b}—, —Rₐ—O—C(O)—O—R_{b}—, —Rₐ—O—C(O)—NR₁₅- R_{b}-(where R₁₅ is a is H or a substituted or unsubstituted C1-C5 alkyl), —Rₐ—CH(OH)—O—R_{b}—, —Rₐ—S—S—R_{b}—, —Rₐ—O—P(O)(OR₁₁)—O—R_{b}— (where R₁₁ is H or a substituted or unsubstituted C1-C5 alkyl), or —Rₐ—C(O)—NR₁₅-R_{b}— (where R is is a is H or a substituted or unsubstituted C₁-C₅ alkyl), where Rₐ and R_{b} are each independently absent or substituted or unsubstituted alkyl. In another embodiment, Rₐ and R_{b} are each independently absent or a C2-C16 substituted or unsubstituted alkyl. In one embodiment of the foregoing, R₆ is a straight chain substituted or unsubstituted C1-C8 alkyl or a branched substituted or unsubstituted C1-C8 alkyl, R₇ is —Rₐ—C(O)—O—R_{b}— and R₈ is absent. In another embodiment of the foregoing, R₆ is a straight chain substituted or unsubstituted C1-C4 alkyl or a branched substituted or unsubstituted C1-C4 alkyl, R₇ is —Rₐ—C(O)—O—R_{b}— and R₈ is absent. In one embodiment of the foregoing, R₆ is, —CH₂—, —CH₂—CH₂—, or —CH₂(CH₃)— and R₇ is —C(O)—O— and R₈ is absent.

In a particular embodiment, R₃ is —C(O)—(CH₂)₃ and R₄ is —CH₂—C(O)—O—, —CH₂—CH₂—C(O)—O— or —CH₂(CH₃)—C(O)—O—.

In a particular embodiment, R₃ is —C(O)—(CH₂)₃ and R₄ is —CH₂—CH₂—O—C(O), —CH₂—CH₂—CH₂—O—C(O), —CH₂—CH₂—CO—NH—(C₆H₄)—O—C(O)—.

In certain aspects of the POZ-lipid conjugates of formulas I to IV, L, L₁, L₂, and L₃ are independently selected for each occurrence from one or more of the cleavable moieties described above. In a particular aspect, when more than one L, L₁, L₂, or L₃ linkage are present in a POZ-Lipid conjugate, L, L₁, L₂, and L₃ are each the same. In another particular aspect, when more than one L, L₁, L₂, or L₃ linkage are present in a POZ-Lipid conjugate, at least one L, L₁, L₂, and L₃ is different from the remaining linkages.

In some embodiments, the POZ-lipid degraded when exposed to plasma enzymes as well as amidases. In particular, ester linkages linking POZ to lipids can be controlled to slow down or speed up cleavage to release lipid from POZ-lipid in plasma. In other embodiments, the POZ-lipid does not degrade in plasma, but does degrade when exposed to amidases that are present in certain tissues. In still other embodiments, the POZ-lipid is stable in plasma and when exposed to amidases. In other words, the LNPs of the present disclosure may include POZ-lipids that are stable in plasma (e.g., amines, amides), POZ-lipids that that can be tuned or controlled to degrade at a desired rate in plasma (e.g., esters), and POZ-lipids that are stable in plasma, but degrade in specific environments. In this regard, substantially precise control of breakdown rate can be provided by carefully selecting the linkage between the POZ and the lipid. In this aspect, the POZ-lipid may have controllable degradability in physiological media. For example, in one embodiment, the controllable degradability of the linking group results in a POZ-lipid that is stable in physiological media. In this aspect, the rate of hydrolysis of the POZ-lipid, i.e., the time it takes to degrade the POZ-lipid (which is usually measured in terms of its half-life), is an indicator of the stability/degradability of the linkage between the POZ and the lipid. In one aspect, the linking group between the POZ polymer and the lipid enables a POZ-lipid with a hydrolysis half-life in 50 percent human plasma of at least about 120 hours.

In another embodiment, the controllable degradability of the linking group results in a POZ-lipid that degrades over time in physiological media. In certain aspects, the linking group between the POZ polymer and the lipid enables a POZ-lipid with a hydrolysis half-life in 50 percent human plasma of about 10 minutes or less. For example, the linking group between the POZ polymer and the lipid may be selected such that the POZ-lipid has a hydrolysis half-life in 50 percent human plasma of about 3 minutes to about 7 minutes. In other aspects, the linking group between the POZ polymer and the lipid is selected such that the POZ-lipid has a hydrolysis half-life in 50 percent human plasma of about greater than about 10 minutes. For example, the linking group between the POZ polymer and the lipid may be selected such that the POZ-lipid has a hydrolysis half-life in 50 percent human plasma of about 11 minutes to about 8 hours. In one embodiment, the POZ-lipid has a hydrolysis half-life in 50 percent human plasma of about 2 hours to about 5 hours.

### Administration

While the LNPs of the present disclosure target antigen-presenting cells, the LNPs of the present disclosure may be delivered to any cell. After in vivo administration of the LNPs, the payload, e.g.. oligonucleotide, is released. In this aspect, the LNPs of the present disclosure may be included a pharmaceutical composition capable of eliciting a treatment for a disorder or disease. For example, pharmaceutical compositions including LNPs made in accordance with the present disclosure may be used to prevent or treat infectious diseases including, but not limited to, SARS-CoV-2, rabies, influenza, and others by specifically targeting certain immune cells other than the antigen-presenting cells. In addition, pharmaceutical compositions including LNPs made in accordance with the present disclosure may be used as therapeutics for cancer and genetic diseases. Such pharmaceutical compositions may also include a pharmaceutically acceptable carrier in addition to the LNPs.

In one embodiment, a pharmaceutical composition including an effective amount of LNP of the present disclosure can be delivered to an animal. In one embodiment, the animal is a human. Delivery of an effective amount of a LNP of the present disclosure may occur via subcutaneous, intravenous, intramuscular, intradermal or aerosol routes. In some embodiments, a pharmaceutical composition including an effective amount of LNP of the present disclosure may be administered intravenously to deliver an encapsulated payload to the liver and spleen. In some embodiments, intravenous administration of the LNP of the present disclosure facilitates delivery of an encapsulated payload to endothelial cells, dendritic cells, Kupffer cells, and/or hepatocyte cells in the liver. In other embodiments, intravenous administration of the LNP of the present disclosure facilitates delivery of an encapsulated payload to macrophages, B cells, T cells, and/or dendritic cells in the spleen. In still other embodiments, intravenous administration of the LNP of the present disclosure facilitates enhanced delivery of an encapsulated payload to dendritic cells and/or macrophages in the spleen (as compared to its PEG-LNP counterpart). For example, the amount of encapsulated payload in dendritic cells in the spleen after intravenous administration of the LNP of the present disclosure may be at least about 15 percent more than the amount of encapsulated payload in dendritic cells in the spleen after administration of a comparable PEG-LNP. Similarly, the amount of encapsulated payload in macrophages in the spleen after intravenous administration of the LNP of the present disclosure may be at least about 10 percent more than the amount of encapsulated payload in macrophage cells in the spleen after administration of a comparable PEG-LNP. Without being bound by any particular theory, it is believed that, not only does a POZ-LNP effectively deliver an encapsulated payload after intravenous injection, it is more effective at delivering the encapsulated payload to antigen-presenting splenic cells, i.e., macrophages and dendritic cells, than its PEG-LNP counterpart.

In other embodiments, a pharmaceutical composition including an effective amount of LNP of the present disclosure may be administered intramuscularly to deliver an encapsulated payload to on-target tissue such as muscle and lumbar aortic lymph nodes, as well as the liver and spleen. In some embodiments, intramuscular administration of the LNP of the present disclosure facilitates delivery of an encapsulated payload to macrophages, dendritic cells, endothelial cells, and/or fibroblasts in muscle. In other embodiments, intramuscular administration of the LNP of the present disclosure facilitates delivery of an encapsulated payload to macrophages, monocytes, B cells, and/or dendritic cells in muscle. In still other embodiments, intramuscular administration of the LNP of the present disclosure facilitates delivery of an encapsulated payload to endothelial cells, dendritic cells, Kupffer cells, and/or hepatocyte cells in the liver. In yet other embodiments, intramuscular administration of the LNP of the present disclosure facilitates delivery of the encapsulated payload to macrophages, B cells, T cells, and/or dendritic cells in the spleen.

In some embodiments, intramuscular administration of the LNP of the present disclosure facilitates enhanced delivery of the encapsulated payload to endothelial cells in the liver (as compared to its PEG-LNP counterpart). For example, the amount of encapsulated payload in endothelial cells in the liver after intramuscular administration of the LNP of the present disclosure may be at least about 20 percent more than the amount of encapsulated payload in endothelial cells in the liver after administration of a comparable PEG-LNP. In some aspects, the amount of encapsulated payload in endothelial cells in the liver after intramuscular administration of the LNP of the present disclosure may be at least about 30 percent more than the amount of encapsulated payload in endothelial cells in the liver after administration of a comparable PEG-LNP.

In other embodiments, intramuscular administration of the LNP of the present disclosure facilitates enhanced delivery of the encapsulated payload to dendritic cells and/or macrophage cells in the spleen (as compared to its PEG-LNP counterpart). For example, the amount of encapsulated payload in dendritic cells in the spleen after intramuscular administration of the LNP of the present disclosure may be at least about 5 percent more than the amount of encapsulated payload in dendritic cells in the spleen after administration of a comparable PEG-LNP. In some embodiments, the amount of encapsulated payload in dendritic cells in the spleen after intramuscular administration of the LNP of the present disclosure may be at least about 10 percent more than the amount of encapsulated payload in dendritic cells in the spleen after administration of a comparable PEG-LNP. Similarly, the amount of encapsulated payload in macrophage cells in the spleen after intramuscular administration of the LNP of the present disclosure may be at least about 5 percent more than the amount of encapsulated payload in macrophage cells in the spleen after administration of a comparable PEG-LNP. Without being bound by any particular theory, it is believed that, not only does a POZ-LNP effectively deliver an encapsulated payload to tissue near the injection site and/or draining lumbar aortic lymph nodes after intramuscular injection, it is more effective at delivering the encapsulated payload to antigen-presenting splenic cells, i.e., macrophages and dendritic cells, and liver endothelial cells than its PEG-LNP counterpart.

Importantly, administration of the LNPs of the present disclosure do not generate as significant of an immune response, including, but not limited to, the generation of IgM antibodies specific to POZ, as compared to the immune response generated by comparable PEG-LNPs. More specifically, the LNPs described herein generate a reduced immune response, including, but not limited to, the generation of IgM and/or IgG antibodies specific to the polymer portion, as compared to a comparable PEG-LNP. In some aspects, after a second administration of a LNP of the present disclosure, the LNP is present in the blood or a tissue of the subject at a concentration of at least 75%, such as 80%, 85%, 90%, 95%, or greater, as compared to the first administration. In other aspects, a LNP of the present disclosure has a reduced accelerated blood clearance after a second dose.

Repeat dosing, i.e., three or more doses each administered a week apart, of a POZ-LNP formed in accordance with the present disclosure results in more particles reaching target cell types than repeat dosing of a comparable PEG-LNP. In some embodiments, the generation of IgM antibodies specific to the polymer portion is at least about 10 percent less after repeat dosing with a POZ-LNP than the comparable PEG-LNP. In other embodiments, the generation of IgM antibodies specific to the polymer portion is at least about 15 percent less after repeat dosing with a POZ-LNP than the comparable PEG-LNP. In still other embodiments, the generation of IgM antibodies specific to the polymer portion is at least about 20 percent less after repeat dosing with a POZ-LNP than the comparable PEG-LNP.

Without being bound by any particular theory, a further reduction in generation of IgM and/or IgG antibodies specific to the polymer portion may be achieved with administration of multiple doses of a POZ-LNP formed in accordance with the present disclosure when the time between doses is greater than seven (7) days/one (1) week. In this aspect, the generation of IgM and/or IgG antibodies specific to the polymer portion is at least about 50 percent less after repeat dosing with a POZ-LNP than the comparable PEG-LNP when the repeat doses are 30 or more days apart. In some embodiments, the generation of IgM and/or IgG antibodies specific to the polymer portion is at least about 60 percent less after repeat dosing with a POZ-LNP than the comparable PEG-LNP when the repeat doses are 30 or more days apart. In other embodiments, the generation of IgM and/or IgG antibodies specific to the polymer portion is at least about 70 percent less after repeat dosing with a POZ-LNP than the comparable PEG-LNP when the repeat doses are 30 or more days apart.

In some aspects, the LNPs of the present disclosure allow for delivery of an encapsulated payload to target tissues of a subject without producing an immune response that promotes accelerated blood clearance (ABC) in response to subsequent doses of the LNP. In particular, a first dose of a LNP of the present disclosure produces an attenuated immune response upon administration of a subsequent dose of LNP, and subsequent doses of LNP to the subject also do not cause the subject to have an ABC response. In other aspects, the LNPs of the present disclosure may not bind to IgM, including but not limited to natural IgM, after repeat dosing where each subsequent dose is administered after a predetermined amount of time.

### EXAMPLES

The following examples do not limit the invention or the claimed subject-matter. Rather, these examples are intended to further illustrate embodiments of the present disclosure.

### Example 1: Formulation of LNPs of the Present Disclosure

LNPs were prepared via microfluidic synthesis in a NanoAssemblr® Ignite™ system with different formulations:
1a. Moderna® LNP Spikevax that includes SM-102 (proprietary ionizable lipid), cholesterol, DSPC (structural lipid) and PEG-1,2-Dimyristoyl-rac-glycerol (DMG);
1b. Moderna® LNP Spikevax that includes SM-102, cholesterol, DSPC, and poly(ethyloxazoline) (PEOZ)-DMG;
2a. Alnylam Onpattro® LNP that includes MC3 (proprietary ionizable lipid optimized for siRNA payloads), cholesterol, DSPC and PEG-DMG; and
2b. Alnylam Onpattro® LNP that includes MC3, cholesterol, DSPC and PEOZ-DMG.

More specifically, mRNA was diluted in 10 mM citrate buffer (Teknova). d-Lin-MC3-DMA (MC3) (MedKoo Biosciences, 555308) or SM-102 (Cayman Chemical Company, 33474), DMG-PEOZ 2,000 or DMG-PEG 2,000 (Avanti, 880151), cholesterol (Avanti, 700100), and distearoylphosphatidylcholine (DSPC) (Avanti, 850365) were diluted in 100% ethanol. Both phases were loaded into separate syringe pumps. The citrate and ethanol phase were mixed in a microfluidic device at a rate of 600 μL/min and 200 μL/min, respectively.

LNP hydrodynamic diameter was measured using high-throughput dynamic light scattering (DLS) (DynaPro Plate Reader II, Wyatt). LNPs were diluted in sterile 1X PBS and analyzed. Encapsulation efficiency was assessed using a RiboGreen Assay (Thermo Fisher Scientific, R11490) with fluorescence read in a VICTOR X4 2030 Multilabel Reader (PerkinElmer).

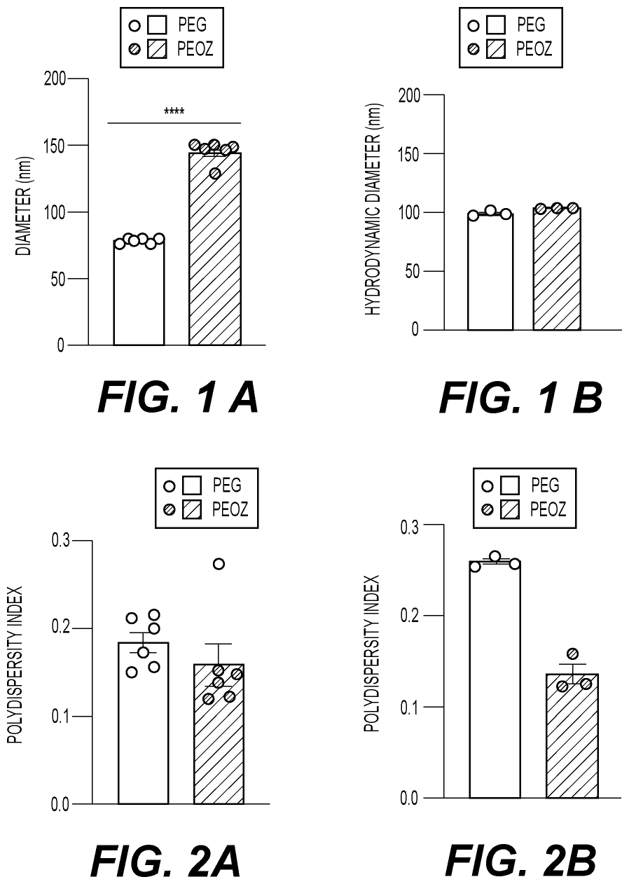

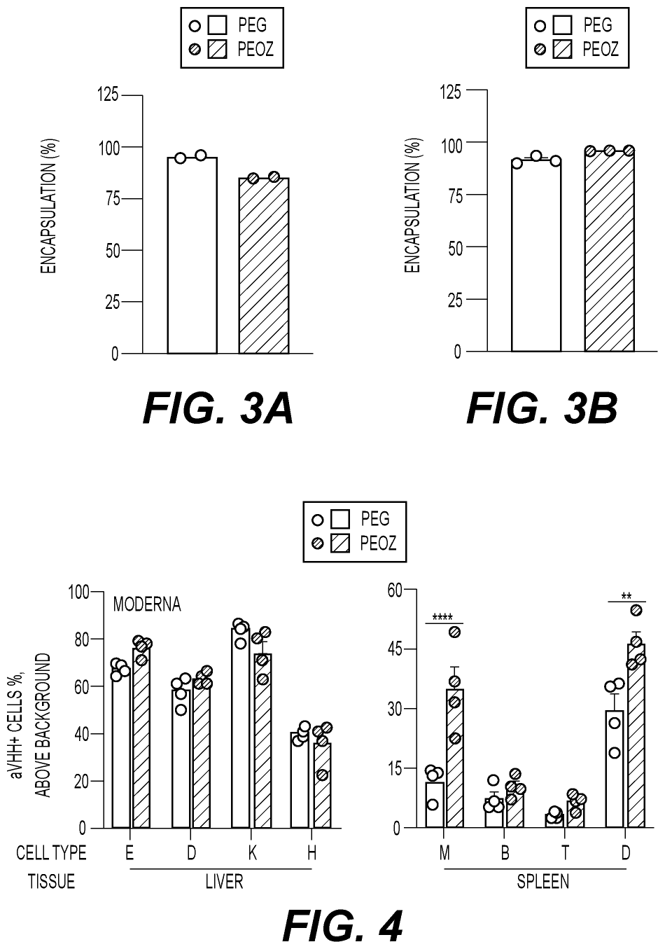

The comparison of the hydrodynamic diameter, polydispersity indices, and encapsulation efficiencies of each comparable LNP (i.e., 1a. vs. 1b. and 2a. vs 2b) demonstrate that POZ-LNPs of the present disclosure have similar biophysical traits to their PEG-LNP counterparts. More specifically, FIG. 1A demonstrates that the hydrodynamic diameter of 1b. (145 nm) is greater than 1a. (78 nm). FIG. 1B demonstrates that the hydrodynamic diameter of 2b. was slightly higher than 2a. FIG. 2A demonstrates that the polydispersity indices were similar for 1a. and 1b. FIG. 2B demonstrates that the polydispersity index of 2a. is greater than 2b. FIG. 3A demonstrates that the encapsulation efficiency of 1a. and 1b were similar. Likewise, FIG. 3B shows that the 2a. and 2b have similar encapsulation efficiencies.

### Example 2: Uptake and Expression of Payload after Intravenous Administration

LNPs 1a.-2b. were each formulated to carry mRNA encoding glycosylphosphatidylinositol (GPI)-anchored camelid VHH antibody (anchored VHH, aVHH+). aVHH+ is an antibody that is recognized specifically by a reporter antibody that is not recognized by any tissues in the mouse and can be used to quantify LNP delivery. Four mice C57BL/6J (Jackson Laboratory)) were used for each group. LNPs were injected via the lateral tail vein at the doses indicated below:
Formulations 1a and 1b: 0.25 mg/kg mouse
Formulations 2a and 2b: 1.00 mg/kg mouse
   PBS-treated mice were used as negative controls. After 24 hours, mouse tissues were then harvested and stained for the presence of aVHH+ as percent above background signal. The tissues were then evaluated for aVHH+ presence in the liver and spleen employing the panel of cell type specific antibodies in Table 1 below:

TABLE 1
Cell Types

Endothelial (E): CD31+
Dendritic (D): CD45+, CD11c+
Macrophages (M): CD45+, CD11b+
Kupffer (K): CD45+, CD68+
B cells (B): CD19+
T cells (T): CD3+
Hepatocytes (H): CD31−/CD45−

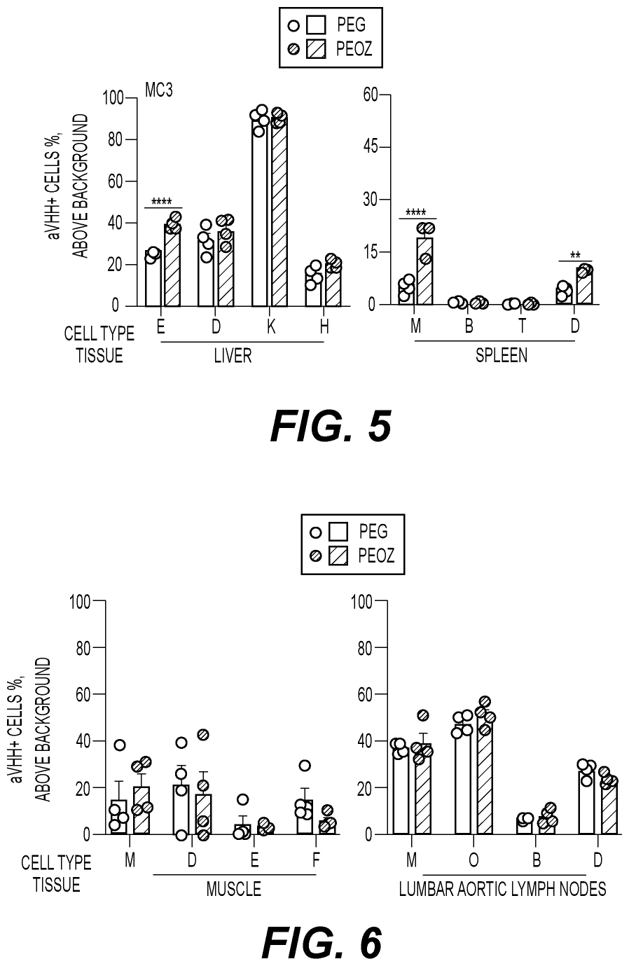

As shown in FIG. 4, most tissues demonstrated no discernible difference in the expression of the payload, e.g., hepatocytes, B cells and T cells in the spleen, and resident dendritic cells in the liver. However, as shown in FIGS. 5, the macrophage and dendritic cell compartments in the spleen showed a dramatic difference when comparing PEG-DMG LNPs and PEG-MC3 LNPs, i.e., Formulations 1a. and 2a., to PEOZ-DMG LNPs and PEOZ-MC3 LNPs, i.e., Formulations 1b. and 2b. In addition, as shown in FIGS. 5A, the liver endothelial cell compartments in the liver showed a significant difference when comparing PEG-MC3 LNPs, i.e., Formulation 2a., to PEOZ-MC3 LNPs, i.e., Formulation 2b. In particular, the expression of the payload in those compartments was independent of the formulation.

The observation of a preferential uptake of PEOZ-DMG LNPs made in accordance with the present disclosure and expression of the payload by antigen-presenting cells in the spleen that is independent of formulation is unexpected. Other cell types found in other tissues (for example endothelial cells, Kupffer cells and hepatocytes) did not demonstrate a preferential uptake when compared to PEG-DMG LNPs. Indeed, other immune cells including B cells and T cells in the spleen did not demonstrate a preferential uptake of PEOZ-DMG LNPs (FIGS. 4B and 5B). The observation was limited to antigen-presenting cells in the macrophage and dendritic cell compartments in the spleen.

### Example 3: Uptake and Expression of Payload after Intramuscular Administration

LNPs 1a.-1b. were again each formulated to carry mRNA encoding aVHH. Four mice C57BL/6J (Jackson Laboratory)) were used for each group. LNPs were injected via the quadricep muscles of both hind legs at the doses indicated below:
Formulations 1a and 1b: 3 μg/hind leg

After 24 hours, mouse tissues were then harvested and stained for the presence of the anchored antibody (aVHH+) as percent above background signal. More specifically, the injected quadriceps were isolated, finely minced, and transferred to 5 mL of a digestive enzyme solution with Collagenase B (Sigma Aldrich, #11088831001), Dispase II neutral protease, grade II (Sigma Aldrich, #04942078001), and RPMI-1640 Medium. Lymph nodes were minced finely in 300 μL of RPMI-1640 Medium to which 700 μL of a digestive enzyme solution with Collagenase D (Sigma Aldrich, #11088866001) and 10% FBS (Sigma Aldrich, #F2442-500ML) and tissues were incubated at 37° C. at 600 rpm for 20 minutes. Spleen tissues were finely minced, then placed in 1×PBS. Liver tissues were finely minced and then placed in a digestive enzyme solution with Collagenase Type I (Sigma Aldrich), Collagenase XI (Sigma Aldrich) and Hyaluronidase (Sigma Aldrich) at 37° C. at 550 rpm for 45 minutes.

The tissues were then evaluated for aVHH+ presence in the muscle, lumbar aortic lymph nodes, liver, and spleen employing the same panel of cell type specific antibodies in Example 2, Table 1.

As shown in FIG. 6, there was no significant difference between aVHH+ cells for on-target organs (i.e., muscle near the injection site draining lumbar aortic nodes). However, as shown in FIG. 7, the endothelial cell compartments in the liver and dendritic cell compartments in the spleen showed a statistically significant difference when comparing PEG-DMG LNPs, i.e., Formulation 1a., to PEOZ-DMG LNPs, i.e., Formulation 1b. In particular, the expression of the payload in those compartments was independent of the formulation.

The observation of a preferential uptake of PEOZ-DMG LNPs made in accordance with the present disclosure and expression of the payload by liver endothelial cells and spleen dendritic cells that is independent of formulation is unexpected.

### Example 4: Uptake and Expression of Payload After Intramuscular Administration

LNPs 1a.-1b. were again each formulated to carry mRNA encoding luciferase to determine the protein expression and immunostimulation after repeat administration.

Four mice C57BL/6J (Jackson Laboratory)) were used for each group. LNPs were injected via the lateral tail vein at the doses indicated below:
Group A: 0.25 mg/kg once at fourth week
Group B: 0.25 mg/kg mouse once per week for four weeks

Tissues were isolated 48 h after administration of LNPs. To measure luminescence, mice were euthanized and organs were collected; organs were submerged in Nano-Glo luciferase assay substrate (Promega, N1110) for 5 min before being placed on solid black paper for imaging. Luminescence was measured using an IVIS imaging system (PerkinElmer) and quantified using LivingImage software (PerkinElmer). To quantify anti-PEG or anti-PEOZ IgM in serum, 100 microliters carboxy-modified latex beads (Life Technologies, C37259) were coupled with 5 mg 1-ethyl-3-(3-dimethylaminopropyl) carbodiimide HCl (TCI Chemicals, D1601-5G) for 15 min at room temperature on a plate shaker at maximum rpm. The activated beads were incubated with 12 μg DMG-PEG 2,000 or DMG-PEOZ 2,000 for 2 hours. Beads were washed two times with PBS, resuspended in 2% MSD Blocker A (Meso Scale Diagnostics, R₉₃AA), and stored at 4° C. overnight. Ten microliter mouse serum samples, isolated 48 h after administration of LNPs, were used at 1:100 dilutions and incubated with the beads at room temperature on a plate shaker for 1 hour. Beads were incubated with Mouse IgM Monoclonal (primary) Antibody in the dark. Samples were washed and incubated with Goat anti-Rat IgG (H+L) Highly Cross-Adsorbed Secondary Antibody, Alexa Fluor Plus 647 (Thermo Fisher Scientific, A48265) for fluorescent detection. After further washing, beads were analyzed by flow cytometry.

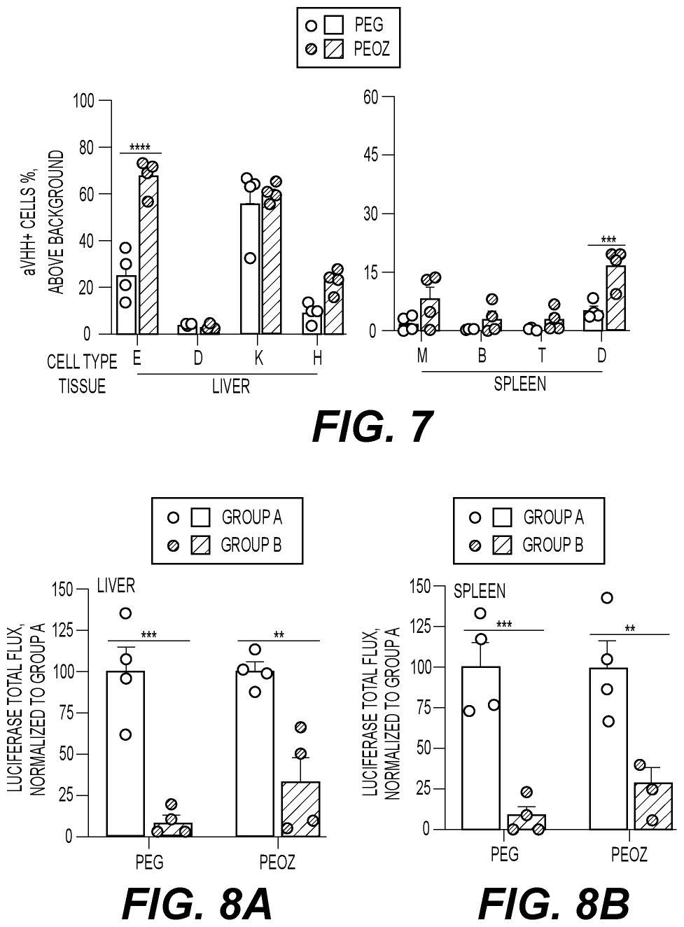

As shown in FIGS. 8A-8B, livers and spleens from Group B mice had significantly less luminescence than livers and spleens from Group A mice. However, as shown in FIG. 8A, the decrease in liver luminescence for Group B mice injected with Formulation 1b. was less (67 percent) than the decrease in liver luminescence for Group B mice injected with Formulation 1a. (91 percent). Similarly, as shown in FIG. 8B, the decrease in splenic luminescence for Group B mice injected with Formulation 1b. was less (71 percent) than the decrease in liver luminescence for Group B mice injected with Formulation 1a. The observation of less of a decrease in liver and splenic luminescence with a PEOZ-LNP (Formulation 1b.) suggests that, while anti-PEOZ antibodies and anti-PEG antibodies are formed in Group B mice injected with repeat doses of Formulation 1b. and Formulation 1a., respectively, the amount of anti-PEOZ antibodies in Group B/Formulation 1b. mice is significantly less than the amount of anti-PEG antibodies in Group B/Formulation 1a. mice after repeat doses.

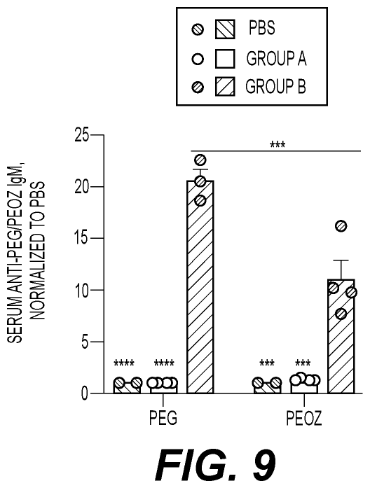

FIG. 9 demonstrates that Group B/Formulation 1b. had significantly less antibodies after repeat dosing than Group B/Formulation 1a.

What is claimed is:

1. A method for preferentially delivering a payload to antigen-presenting cells in a subject comprising:
providing a lipid nanoparticle encapsulating the payload, wherein the lipid nanoparticle comprises:
a POZ-lipid of Formula I: 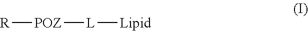
wherein R comprises an initiating group,
POZ comprises poly(ethyloxazoline),
L comprises a physiologically degradable linking group, and
Lipid comprises a non-charged lipid comprising at least one
hydrophobic moiety;
an ionizable or cationic lipid;
a helper lipid;
a sterol lipid; and
administering an effective amount of the lipid nanoparticle to the subject.

2. The method of claim 1, wherein the antigen-presenting cells comprise splenic macrophage cells, dendritic cells, or combinations thereof.

3. The method of claim 1, wherein POZ comprises [N(COR₂)CH₂CH₂]ₙ, where R₂ is ethyl.

4. The method of claim 1, wherein R comprises a hydrogen, a substituted or unsubstituted alkyl, an alkyne-substituted alkyl, a triazole with attached carboxylic acid, or a substituted or unsubstituted aralkyl group.

5. The method of claim 1, wherein L comprises ethers, esters, carboxylate esters, carbonate esters, carbamates, amines, amides, urethanes, disulfides, and combinations thereof.

6. The method of claim 1, wherein Lipid comprises two hydrophobic moieties.

7. The method of claim 1, wherein Lipid comprises phospholipid, a glycerolipid, a di-alkyl acetamide, or a combination thereof.

8. The method of claim 1, wherein Lipid comprises 1,2-dimyristoyl-sn-glycerol, 1,2-dilauroyl-sn-glycerol, or a combination thereof.

9. The method of claim 1, wherein the payload comprises an oligonucleotide.

10. A method of delivering a payload to target tissue of a subject, comprising:
providing a lipid nanoparticle encapsulating the payload, wherein the lipid nanoparticle comprises:
a POZ-lipid of Formula I: 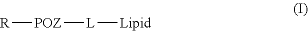
wherein R comprises an initiating group,
POZ comprises poly(ethyloxazoline),
L comprises a physiologically degradable linking group, and
Lipid comprises a non-charged lipid comprising at least one hydrophobic moiety;
an ionizable or cationic lipid;
a helper lipid;
a sterol lipid; and
administering an effective amount of the lipid nanoparticle to the subject.

11. The method of claim 10, wherein the step of administering comprises intramuscularly injecting the subject.

12. The method of claim 10, wherein the step of administering comprises intramuscularly injecting the subject with a plurality of doses, wherein after a first dose is administered, each subsequent dose is administered after a predetermined amount of time.

13. The method of claim 12, wherein the predetermined amount of time is at least 30 days.

14. The method of claim 10, wherein the target tissue comprises liver, spleen, or a combination thereof.

15. The method of claim 10, wherein POZ comprises [N(COR₂)CH₂CH₂]ₙ, where R₂ is ethyl.

16. The method of claim 10, wherein R comprises a hydrogen, a substituted or unsubstituted alkyl, an alkyne-substituted alkyl, a triazole with attached carboxylic acid, or a substituted or unsubstituted aralkyl group.

17. The method of claim 10, wherein L comprises ethers, esters, carboxylate esters, carbonate esters, carbamates, amines, amides, urethanes, disulfides, and combinations thereof.

18. The method of claim 10, wherein Lipid comprises phospholipid, a glycerolipid, a di-alkyl acetamide, or a combination thereof.

19. The method of claim 10, wherein Lipid comprises 1,2-dimyristoyl-sn-glycerol, 1,2-dilauroyl-sn-glycerol, or a combination thereof.

20. The method of claim 10, wherein the payload comprises mRNA.

21. A method for delivering a therapeutic level of an immunogenic payload to target tissue of a subject, comprising:
administering a plurality of doses of a lipid nanoparticle to the subject, wherein the lipid nanoparticle encapsulates an mRNA encoding an antigen, wherein the lipid nanoparticle comprises an ionizable lipid, a helper lipid, a structural lipid, and a POZ-lipid of Formula I: 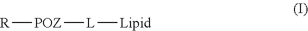
wherein R comprises an initiating group,
POZ comprises poly(ethyloxazoline),
L comprises a physiologically degradable linking group, and
Lipid comprises a non-charged lipid comprising at least one hydrophobic moiety, and
wherein the LNP induces an attenuated drug response associated with an LNP.

22. The method of claim 21, wherein the drug response comprises an attenuated induction of IgM, attenuated induction of IgG, an attenuated accelerated blood clearance, or a combination thereof.

23. The method of claim 21, wherein the target tissue comprises liver, spleen, or a combination thereof.

24. The method of claim 21, wherein POZ comprises [N(COR₂)CH₂CH₂]ₙ, where R₂ is ethyl.

25. The method of claim 21, wherein R comprises a hydrogen, a substituted or unsubstituted alkyl, an alkyne-substituted alkyl, a triazole with attached carboxylic acid, or a substituted or unsubstituted aralkyl group.

26. The method of claim 21, wherein L comprises ethers, esters, carboxylate esters, carbonate esters, carbamates, amines, amides, urethanes, disulfides, and combinations thereof.
